SEQUENCE LISTING (4 sequences)

SEQ ID NO: 1 - protein, 33 aa, Triticum sp.
LQLQPFPQPQLPYPQPQLPYPQPQLPYPQPQPF

SEQ ID NO: 2 - protein, 5 aa, synthetic construct
PQLPF

SEQ ID NO: 3 - protein, 5 aa, synthetic construct
GGGGS

SEQ ID NO: 4 - protein, 14 aa, synthetic construct
GSTSGSGKSSEGKG

(19) United States
(12) Patent Application Publication
(10) Pub. No.: US 2026/0364210 A1
(43) Pub. Date: Nov. 19, 2026

(54) COMPOSITIONS AND METHODS FOR TRANSGLUTAMINASE-MEDIATED ENDOCYTOSIS

(71) Applicant: The Board of Trustees of the Leland Stanford Junior University, Stanford, CA (US)

(72) Inventors: Chaitan Khosla, Stanford, CA (US); Elise Loppinet, Redwood City, CA (US); Harrison Besser, Palo Alto, CA (US)

(21) Appl. No.: 19/105,006

(22) PCT Filed: Jul. 5, 2023
(86) PCT No.: PCT/US2023/026914

§ 371 Date: Feb. 19, 2025

Related U.S. Application Data
(60) Provisional application No. 63/373,947, filed on Aug. 30, 2022.
(60) Provisional application No. 63/431,570, filed on Dec. 9, 2022.

(51) Int. Cl. A61K 49/00; A61K 47/64

(52) U.S. Cl. CPC A61K 49/0056; A61K 47/64; A61K 49/0032

(57) ABSTRACT

Compositions and methods are provided for in vivo or ex vivo delivery of a molecular cargo into endo-lysosomal compartments of LRP1-expressing cells. A molecular cargo of interest is linked to a substrate or inhibitor for recognition and transport via human transglutaminase 2 (TG2).

### GOVERNMENT RIGHTS

This invention was made with Government support under contract DK063158 awarded by the National Institutes of Health. The Government has certain rights in this invention.

### BACKGROUND OF THE INVENTION

Transglutaminase 2 (TG2) is a member of the human transglutaminase family of enzymes, which is abundantly expressed in various tissues and is found in both intra- and extra-cellular locations (Lorand and Graham, 2003). It possesses the catalytic activity of deamidating glutamine sidechains on substrate peptides or proteins or crosslinking them with biogenic small molecule or protein-bound amines. An example of a high-affinity TG2 substrate is SEQ ID NO:1, LQLQPFPQPQLPYPQPQLPYPQPQLPYPQPQPF, a 33-mer gluten peptide that reveals HLA-DQ2 epitopes upon TG2-catalyzed deamidation at the underlined glutamine residues (Shan et al., 2002).

TG2 has been attributed with various biological functions (for reviews, see e.g. lismaa et al., 2009; Nurminskaya & Belkin, 2012). It has also been implicated in the pathogenesis of many human diseases, particularly inflammatory disorders such as sepsis (Falasca et al., 2008), renal fibrosis (Chen et al., 2018) and celiac disease (Abadie et al., 2020). Many classes of TG2 inhibitors have been engineered as research tools (Keillor et al., 2015). Examples relevant to this invention include irreversible inhibitors harboring the electrophilic 6-diazo-5-oxo-L-norleucine (Hausch et al., 2003) or the α,β-unsaturated sulfone, ester or amide (Büchold et al., Cells 2022, 11:1667; Campbell et al., 2020 PCT/US2019/045827) moieties.

### SUMMARY OF THE INVENTION

Provided herein are compositions and methods for in vivo or ex vivo delivery of a molecular cargo into endo-lysosomal compartments of LRP1-expressing cells. The molecular cargo is linked to a substrate or inhibitor for recognition and transport via human transglutaminase 2 (TG2). The compositions of the disclosure can be incorporated into formulations for therapeutic and diagnostic applications. The compositions also provide a flexible platform for delivery of a cargo of interest to lysosomal compartments.

The disclosure herein demonstrates a novel biological mechanism by which a TG2 substrate or inhibitor can undergo receptor-mediated endocytosis in a TG2-dependent manner. TG2 binds to gluten and gluten-like molecules in the extracellular matrix. The enzyme-substrate/inhibitor complex is recognized by the plasma protein x2-macroglobulin, and the resulting ternary complex is endocytosed via recognition by the cluster I/II of LRP1 (low-density lipoprotein receptor-related protein-1). Following endocytosis, the TG2 substrate/inhibitor and linked cargo can be delivered to lysosomes, and may be presented on the cell-surface in an MHC-restricted context. The methods and compositions disclosed herein utilize this pathway to provide for enhanced endo-lysosomal uptake by cells expressing LRP1 of molecular cargo linked to a TG2 substrate or inhibitor.

In an embodiment, the TG2 substrate/inhibitor comprises or consists of a polypeptide comprising the pentapeptide sequence Pro-X-Y-Pro-R (formula I), where X is selected from amino acids that engage TG2 through the formation of a covalent enzyme-compound intermediate, including without limitation glutamine, a-diazoketones, a-halo-ketones, αβ-unsaturated carbonyl compounds, and αβ-unsaturated sulfones (see formula II, amino acid X).

Y is any amino acid, including unnatural amino acids with easily appended functional groups, including without limitation primary/secondary amines, alcohols, and carboxylic acids;
R is a natural or non-natural aromatic amino acid, e.g. tyrosine (Y), phenylalanine (F), and tryptophan (W), naphthalene, etc.;
The C-terminus of the pentapeptide can be a carboxylic acid, an ester, or an amide; wherein molecular cargo is linked to the C-terminus of the pentapeptide, and/or to Y. The cargo may be joined to the pentapeptide through a linker, through covalent direct conjugation, through non-covalent high affinity pairing, and the like.

Molecular cargo include any compound that is of interest for delivery to a cell. Cargo moieties of interest include, without limitation, antibiotics, antiviral agents, chemotherapeutic agents, nucleosides, polynucleotides, e.g. siRNA, mRNA, DNA, etc., proteins, fluorescent/radioactive/optical imaging agents, peptides/proteins/enzymes, nucleic acids (siRNA/RNA/DNA/etc.), metal based compounds/catalysts, polymers, site-specific cellular targeting agents (compounds/ligands/antibodies/etc.), immunomodulatory drugs, antigen binding moieties, antibody binding moieties, etc., and are useful for diverse applications such as imaging, treating infection, chemotherapeutic agents, smart adjuvants, gene therapy vectors, biosensors, bioreactors, and so forth. Molecular cargo can be joined to the substrate/inhibitor through a linker, optionally a cleavable linker. In some embodiments, a TG2 substrate/inhibitor as disclosed herein is provided comprising linker as molecular cargo, where the linker can be activated to join the substrate/inhibitor to an agent of interest.

In one embodiment, the TG2 substrate/inhibitor comprises or consists of Formula II:

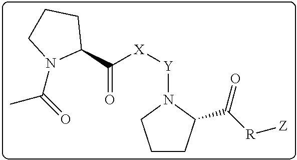

where X is selected from:

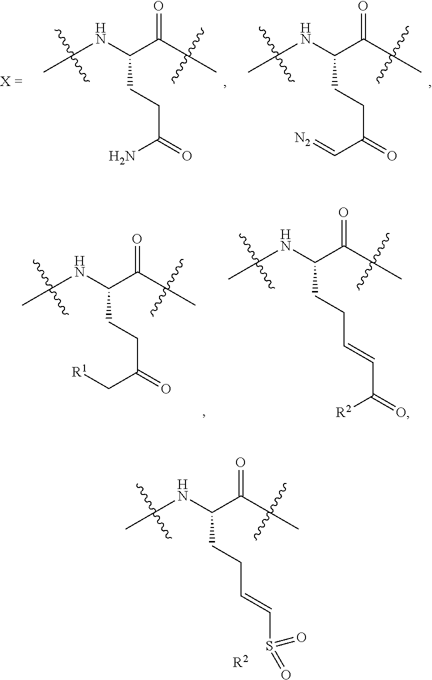

where R¹ is selected from Cl, Br, I, OSO₂CF₃; R² is selected from N(CH₃)₂, OCH₃, NH₂, CH₃, etc.;
Y is any amino acid,

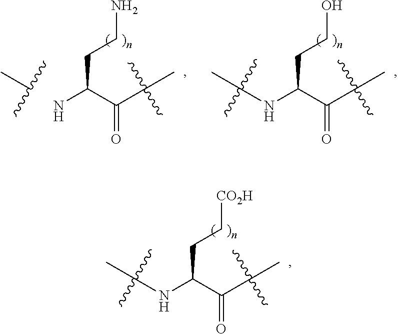

or molecular cargo, or a linker. In some embodiments Y is Leu (L);
R is an aryl, heteroaryl or substituted aryl group. In some embodiments R is Ph, CH₂Ph (Phe, F), CH₂PhOH (Tyr, Y), CH₂Naphthelene;
   and
Z is OH, OR³, NH₂, NHR³, N(R³)₂, etc., or Z is molecular cargo or a linker. R³ is an alkyl, e.g. a lower alkyl, substituted alkyl, heteroalkyl, etc.

The compounds disclosed herein, i.e. a TG2 substrate or inhibitor optionally joined to a molecular cargo directly or through a suitable linker, can be provided as a formulation in combination with a pharmaceutically acceptable carriers, diluents, excipients and/or adjuvants. Formulations may be provided for oral, parenteral, etc. administration. Formulations may be provided in a unit dosage, e.g. in an effective dose for imaging, therapy, and the like.

In some embodiments, compositions and methods are provided for imaging TG2 in vitro or in vivo, the method comprising contacting a subject, tissues, cells, biological fluids, etc. suspected of comprising catalytically active TG2 with an effective dose of a TG2 substrate or inhibitor joined to molecular cargo for imaging, where imaging moieties include without limitation radiography moieties, e.g. heavy metals and radiation emitting moieties, positron emitting moieties, magnetic resonance contrast moieties, and optically visible moieties, e.g., fluorescent or visible-spectrum dyes, visible particles, etc. The imaging moiety is then detected in the tissues, cells, etc. In some embodiments the imaging is performed in vivo. The TG2 substrate or inhibitor and cargo allow for direct visualization of catalytically active TG2 in vivo. The electrophilic amino acid X is selected to comprise an isosteric unnatural amino acid harboring an α,β-unsaturated dimethylamide “soft” electrophile. The resulting non-hydrolyzable adduct thus labels TG2 directly for analysis. Such TG2 targeting molecules are useful in tracking TG2 in a myriad of physiological and pathological conditions both in vitro and in vivo. The imaging composition is used in, for example, the diagnosis or post-diagnostic management of celiac disease, a lifelong autoimmune disorder in which TG2 activity is upregulated and is known to play a critical role in disease pathogenesis. Because of the agent's ability to irreversibly inactivate TG2, it is also expected to deliver therapeutic benefit when chronically administered to a celiac disease patient. Notably, because the same composition of matter has both diagnostic and therapeutic utility, it can enhance disease management by allowing the physician to dynamically adapt the drug dosing regimen to individual patients' changing needs.

In an embodiment, compositions and methods are provided for the targeted proteolysis of a polypeptide, the methods comprising contacting a subject, tissues, cells, biological fluids, etc. comprising a polypeptide targeted for proteolysis, with an effective dose of a TG2 substrate or inhibitor linked to a binding domain that specifically binds to the targeted polypeptide. Specific binding domains of interest include, without limitation, ligands of the targeted polypeptide, antigen binding domains such as antibodies, nanobodies, etc. The targeted polypeptide binds to the substrate/inhibitor, and in the presence of α2-macroglobulin (α2M), this complex undergoes LRP1-mediated endocytosis followed by proteolytic destruction in the lysosome. Such methods are useful in accelerating clearance of targeted pathogenically relevant extracellular proteins, e.g. to reduce its contribution to disease pathogenesis.

In an embodiment, compositions and methods are provided for the delivery of molecular cargo into endo-lysosomal compartments of LRP1-expressing cells, the method comprising contacting a subject, tissues, cells, biological fluids, etc. with an effective dose of a TG2 substrate or inhibitor linked to a cargo for delivery, including without limitation small molecule drugs such as chemotherapeutics, antibiotics, anti-viral agents, etc. The cargo may be joined to the TG2 substrate/inhibitor through a linker. The linker may be a linker that is cleaved in the lysosomal complex, thereby releasing the cargo. The methods may provide for treatment of cancer by the delivery of a chemotherapeutic agent. The methods may provide for treatment of an infection by delivery of an anti-viral or antibiotic agent.

In an embodiment, compositions and methods are provided for the delivery of IgG polypeptides and IgG linked polypeptides into endo-lysosomal compartments of LRP1-expressing cells, the method comprising contacting a subject, tissues, cells, biological fluids, etc. with an effective dose of a TG2 substrate or inhibitor linked to an IgG-binding moiety. The IgG binding moiety can bind to IgG proteins present in the blood or tissue of interest for delivery to the endo-lysosomal compartment, and is useful in treatment to remove maladaptive IgG proteins, e.g in rheumatoid arthritis, ITP, SLE, etc. Alternatively, the IgG binding moiety can be pre-complexed with a compound of interest linked to an IgG, for delivery of the compound of interest to the endo-lysosomal compartment. Such methods can provide for treatment, for example, of disorders of endolysosomal metabolism such as lysosomal storage diseases.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1: Kinetic analysis of TG2 inhibition by HB-225. Both substrate (PQLPF) and inhibitor concentrations were varied.

FIG. 2: Comparison of TG2 inhibition by HB-225 and HB-230. The two compounds have very similar kinetic properties.

FIG. 3A-3C: (A) HB-230 is robustly taken up in the presence (right) but not in the absence (left) of 100 μg/mL of α2M while (B) HB-258 is not and remains mainly in small puncta under the same assay conditions. (C) Fluorescently tagged α2M colocalizes strongly with HB-230.

FIG. 4A-4B: (A) HB-230 (red) does not appear in early endosomes as stained by EEA1 (green) whereas (B) it colocalizes strongly with the lysosomal marker LAMP-1 (green).

FIG. 5A-5C: (A) HB-225 induces a dose dependent uptake of fluorescently tagged α2M. (B) 10 M of CK805 is not capable of causing internalization of α2M despite binding the TG2 active site, and (C) 100 μM cystamine also does not promote uptake of α2M.

FIGS. 6A-6B: Fluorescently tagged 33 mer peptide (A) induces a similar pattern of internalization of α2M as HB-230 (B).

FIG. 7A-7D: (A) Endocytosis of α2M (green) and 33 mer gluten peptide (red) by NRK cells. Inhibitors of clathrin mediated endocytosis including (B) 12.5 mM MβCD, (C) 20 uM Dyno-4a, (D) 25 uM Pitstop abrogate the appearance of large puncta harboring both fluorescently labeled probes.

FIG. 8A-8B: (A) Wild-type MEFs also readily internalize HB-230 and α2M in the presence of exogenous TRX and betamethasone. (B) Under the same conditions LRP-1 knock-out MEFs show markedly reduced uptake of either probe in the form of large puncta.

FIG. 9: Schematic representation of proposed pathway.

FIG. 10A-10B: (A) Streptavidin does not enter the cell in the absence of HB-2110. (B) When co-treated with HB-2110, streptavidin strongly colocalizes with α2M.

FIG. 11: (A) Treating NRK cells with increasing concentrations of HB-3-003 in the presence of fluorescently tagged streptavidin leads to increased internalization of streptavidin after a 90 min treatment. (B) Quantification of dose response. (C) Biotin and PQLPF alone do not lead to increased uptake of streptavidin.

FIG. 12A-12C: (A) Treating NRK cells with increasing concentrations of HB-3-002 leads to decrease of cell membrane cubilin after a 90 min treatment. (B) Quantification of dose response. (C) Vitamin B₁₂ and PQLPF alone do not lead to increased uptake of cubilin.

FIG. 13A-13B: (A) HB-2108 is strongly endocytosed and cleaved leading to diffuse staining, (B) Inhibiting Cathepsin B mitigates diffuse pattern.

FIGS. 14A-14B. Metabolic stability of HB-230 and HB-2121. (A) HB-230 is transformed by chymotrypsin to give the C-terminal carboxylic acid. This metabolite is subsequently cleaved by carboxypeptidase to give the tetrapeptide without the terminal phenylalanine. (B) HB-2121 is stable to both chymotrypsin and carboxypeptidase mediated metabolism.

FIG. 15. Uptake of HB-2121. NRK cells were treated with 2 μM HB-2121 (red) with or without 100 μg/mL α-2-macrogloblulin, showing α2M-enhanced endocytic uptake of the TG2 probe, as was seen with HB-230.

### DETAILED DESCRIPTION OF THE EMBODIMENTS

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the terms “determining,” “measuring,” “assessing,” and “assaying” are used interchangeably and include both quantitative and qualitative determinations.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome. In one example, a physician may attempt to predict the likelihood that a patient will survive.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of cancer in a mammal, particularly in a human, and includes: (a) inhibiting the disease, i.e., arresting its development; and (b) relieving the disease or its symptoms, i.e., causing regression of the disease or its symptoms.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of engineered cells to prevent or delay, to alleviate, or to arrest or inhibit development of the symptoms or conditions associated with disease or other diseases. The term “therapeutic effect” refers to the reduction, elimination, or prevention of the disease, symptoms of the disease, or side effects of the disease in the subject.

As used herein, a “therapeutically effective amount” refers to that amount of the therapeutic agent sufficient to treat or manage a disease or disorder. A therapeutically effective amount may refer to the amount of therapeutic agent sufficient to delay or minimize the onset of disease. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

The terms “specific binding,” “specifically binds,” and the like, refer to non-covalent or covalent preferential binding to a molecule relative to other molecules or moieties in a solution or reaction mixture (e.g., an antibody specifically binds to a particular polypeptide or epitope relative to other available polypeptides). In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity being correlated with a lower Kd.

The term “Alkyl” refers to a C₁-C₂₀ alkyl that may be linear, branched, or cyclic. “Lower alkyl”, as in “lower alkyl”, or “substituted lower alkyl”, means a C₁₋₁₀ alkyl. The term “alkyl”, “lower alkyl” or “cycloalkyl” includes methyl, ethyl, isopropyl, propyl, butyl, isobutyl, sec-butyl, tert-butyl, pentyl, neopentyl, hexyl, cyclopropyl, cyclopropylmethyl, cyclobutyl, cyclobutylmethyl, cyclopentyl, cyclopentylmethyl, cyclohexyl, cyclohexylmethyl, C₆ to C₁₂ spirocycles, cyclopropylethyl, cyclobutylethyl, decalinyl, Bicyclo-[1.1.1]-pentyl, norboranyl, bicylo-[2.2.2]-octyl, cubyl, adamantanyl and related cage hydrocarbon moieties. In certain embodiments, the alkyl is a C₁-C₂₀ alkyl. In certain embodiments the alkyl group is poly deuterated.

A “substituted alkyl” is an alkyl which is typically mono-, di-, or tri-substituted with heterocycloalkyl, aryl, substituted aryl, heteroaryl, nitro, cyano (also referred to herein as nitrile), azido, halo, —OR, —SR, —SF₅, —CHO, —COR, —C(O)OR, —C(O)—NR₂, —OC(O)R, —OC(O)NR₂, —OC(O)OR, —P(O)(OR)₂, —OP(O)(OR)₂, —NR₂, —N⁺R₃ (wherein a counterion may be present), —CONR₂, —NRCOR, —NHC(O)OR, —NHC(O)NR₂, —NHC(NH)NR₂, SO₃, —SO₂OR, —OSO₂R, —SO₂NR₂, or —NRSO₂R, where each R is, independently, hydrogen, lower alkyl, R′-substituted lower alkyl, aryl, R′-substituted aryl, heteroaryl, heteroaryl(alkyl), R′-substituted aryl(alkyl), or aryl(alkyl) and each R′ is, independently, hydroxy, halo, alkyloxy, cyano, thio, SF₅, nitro, alkyl, halo-alkyl, or amino. Substituted alkyls which are substituted with one to three of the substituents selected from the group consisting of alkynyl, cyano, halo, alkyloxy, thio, nitro, amino, or hydroxy are particularly of interest.

The term “Aryl” refers to an aromatic ring having (4n+2) pi electrons that may contain 6 to 20 ring carbon atoms, and be composed of a single ring (e.g., phenyl), or two or more condensed rings, such as 2 to 3 condensed rings (e.g., naphthyl), or two or more aromatic rings, such as 2 to 3 aromatic rings, which are linked by a single bond (e.g., biphenylyl). In certain cases, the aryl is C₆-C₁₆ or C₆ to C₁₄. In certain embodiments the alkyl group has one or more hydrogen atoms replaced with deuterium.

Heteroaryl means an aromatic ring system containing (4n+2)pi electrons and comprised of 1 to 10 ring carbon atoms and 1 to 5 heteroatoms selected from O, N, S, Se, having a single ring (e.g., thiophene, pyridine, pyrazine, imidazole, oxazole, tetrazole, etc.), or two or more condensed rings, for example 2 to 3 condensed rings (e.g., indole, benzimidazole, quinolone, quinoxaline, phenothiazine, etc.), or two or more aromatic rings, such as 2 to 3 aromatic rings, which are linked by a single bond (e.g., bipyridyl). In some cases, the heteroaryl is C₁-C₁₆, and a selection of 1 to 5 heteroatoms consisting of S, Se, N, and 0.

The term “heterocycloalkyl”, “heterocycle”, “heterocyclic group” or “heterocyclyl” refers to a saturated or unsaturated nonaromatic ring system containing 1 to 10 ring carbon atoms and 1 to 5 heteroatoms selected from O, N, S, Se, having a single ring (e.g., tetrahydrofuran, aziridine, azetidine, pyrrolidine, piperidine, tetrathiopyran, hexamethylene oxide, oxazepane, etc.), or two or more condensed rings, such as 2 to 3 condensed rings (e.g., indoline, tetrahydrobenzodiazapines, etc., including fused, bridged and spiro ring systems, having 3-15 ring atoms, included 1 to 4 heteroatoms. In certain cases, the heterocycloalky is C₁-C₁₆, and a selection of 1 to 5 heteroatoms consisting of S, Se, N, and O. In fused ring systems, one or more of the rings can be cycloalkyl, heterocycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

Examples of heterocycles and heteroaryls include, but are not limited to, azetidine, pyrrole, imidazole, benzimidazole, pyrazole, benzopyrazole, tetrazole, 1,2,3-triazole, benzotriazole, 1,2,4-triazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, benzoisothiazole, phenazine, isoxazole, benzoisooxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, benzothiazole, thiazolidine, furan, benzofuran, thiophene, benzothiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, benzotetrahydrofuranyl, and the like.

Substituted heterocycloalkyl, aryl, heteroaryl are optionally substituted with, hydrogen, 1 to 3 alkyl, alkenyl, alkynyl, cycloalkyl, cycloalkyl(alkyl), aryl, substituted aryl, aryl(alkyl), —SO₂NR⁵R⁵, —PO₃H₂, —NRSSO₂R⁶ or —NR⁵C(═O)R⁶, wherein R⁵ and R⁶ are independently, hydrogen, alkyl, alkenyl, alkynyl, cycloalkyl, cycloalkyl(alkyl), aryl, optionally substituted heterocycloalkyl, aryloxy, heteroaryl, heteroaryl(alkyl), or R⁵ and R⁶ together are —(CH₂)₃₋₆— or —(CH₂)₀₋₃X(CH₂)₀₋₃— where X═NR, O, S, SO₂, substituted aryl(alkyl), halo(alkyl), SF₅, NR⁵₃_{⁺}, azido, cyano (also referred to herein as nitrile), —OR⁵, —SR⁵, —NR⁵R⁶, halogen, nitro, SCH₃, OCF₃, SO₂CH₃, SCF₃, SO₂CF₃, CF₃, —SO₂OR⁵, —OSO₂R⁵, CCl₃, —C(═O)R⁵, —C(═O)OR⁵; —C(═O)NR⁵R⁶, —OC(═O)R⁵.

By “substituted” as in “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents. Examples of such substituents include, without limitation, functional groups, and the hydrocarbyl moieties C1-C24 alkyl (including C1-C18 alkyl, further including C1-C12 alkyl, and further including C1-C6 alkyl), C2-C24 alkenyl (including C2-C18 alkenyl, further including C2-C12 alkenyl, and further including C2-C6 alkenyl), C2-C24 alkynyl (including C2-C18 alkynyl, further including C2-C12 alkynyl, and further including C2-C6 alkynyl), C5-C30 aryl (including C5-C20 aryl, and further including C5-C12 aryl), and C6-C30 aralkyl (including C6-C20 aralkyl, and further including C6-C12 aralkyl). The above-mentioned hydrocarbyl moieties may be further substituted with one or more functional groups or additional hydrocarbyl moieties such as those specifically enumerated. Unless otherwise indicated, any of the groups described herein are to be interpreted as including substituted and/or heteroatom-containing moieties, in addition to unsubstituted groups.

“Sulfonyl” refers to the group SO₂-alkyl, SO₂-substituted alkyl, SO₂-alkenyl, SO₂— substituted alkenyl, SO₂-alkynyl, SO₂-substituted alkynyl, SO₂-cycloalkyl, SO₂-substituted cycloalkyl, SO₂-cycloalkenyl, SO₂-substituted cylcoalkenyl, SO₂-aryl, SO₂-substituted aryl, SO₂— heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—. Sulfonimidoyl refers to S(O)(NH)-bonded as for sulfonyl defined above.

The term “water-soluble group” refers to a functional group that is well solvated in aqueous environments and that imparts improved water solubility to the compound to which it is attached. Water-soluble groups of interest include, but are not limited to, polyalcohols, straight chain or cyclic saccharides, primary, secondary, tertiary, or quaternary amines and polyamines, sulfate groups, sulfonate groups, sulfinate groups, carboxylate groups, phosphate groups, phosphonate groups, phosphinate groups, ascorbate groups, glycols, including polyethylene glycols (PEG) and modified PEGs, and polyethers. In some instances, water-soluble groups are primary, secondary, tertiary, and quaternary amines, carboxylates, phosphonates, phosphates, sulfonates, sulfates, —N(H)₀₋₁(CH₂CH₂OH)₁₋₂, —NHCH₂CH₂N(CH₃)₂₋₃, —NHCH₂CH₂SO₃H, —NHCH₂CH₂PO₃H₂ and —NHCH₂CH₂CO₂H, —(CH₂CH₂O)_{yy}CH₂CH₂XR^{yy}, —(CH₂CH₂O)_{yy}CH₂CH₂X—, —X(CH₂CH₂O)_{yy}CH₂CH₂—, glycol, oligoethylene glycol, and polyethylene glycol, wherein yy is selected from 1 to 1000, X is selected from 0, S, and NR^{ZZ}, and R^{ZZ} and R^{YY} are independently selected from H and C1-3 alkyl.

The term “carboxy isostere” refers to standard medicinal bioisosteric replacement groups for carboxylic acids, amides and ester. These include, but are not limited to: acyl cyanamide, tetrazoles, hydroxychromes, 3-hydroxy-1,2,4-triazoles, 1-hydroxy pyrazoles, 2,4-dihydroxy imidazoles, 1-hydroxy imidazole, 1-hydroxy 1,2,3-triazole, alkylsulfonyl carboxamides, hydroxy isoxazoles, 5-hydroxy 1,2,4-oxadiazoles, thiazoles, 1,2,4-oxadiazoles, 1,2,4-oxadiazolones, oxazoles, triazoles, thiazoles, others hydroxamic acids, sulfonimide, acylsulfonamide, sulfonylureas, oxadiazolone, thiazolidinediones, oxadiazole, thiadiazole, isothiazoles, difluorophenols, tetramic acids, tetronic acids, squaric acids, hydroxyquinoline-ones, hydroxyquinoline-2-ones, boronic acids and phosphoric acids.

By the term “functional groups” is meant chemical groups such as halo, hydroxyl, sulfhydryl, C1-C24 alkoxy, C2-C24 alkenyloxy, C2-C24 alkynyloxy, C5-C20 aryloxy, acyl (including C2-C24 alkylcarbonyl (—CO-alkyl) and C6-C20 arylcarbonyl (—CO-aryl)), acyloxy (—O-acyl), C2-C24 alkoxycarbonyl (—(CO)—O-alkyl), C6-C20 aryloxycarbonyl (—(CO)—O-aryl), halocarbonyl (—CO)—X where X is halo), C2-C24 alkylcarbonato (—O—(CO)—O-alkyl), C6-C20 arylcarbonato (—O—(CO)—O-aryl), carboxy (—COOH), carboxylato (—COO—), carbamoyl (—(CO)—NH₂), mono-substituted C1-C24 alkylcarbamoyl (—(CO)—NH(C1-C24 alkyl)), di-substituted alkylcarbamoyl (—(CO)—N(C1-C24 alkyl)₂), mono-substituted arylcarbamoyl (—(CO)—NH-aryl), thiocarbamoyl (—(CS)—NH₂), carbamido (—NH—(CO)—NH₂), cyano (—C≡N), isocyano (—N+≡C—), cyanato (—O—C═N), isocyanato (—O—N+≡C—), isothiocyanato (—S—C═N), azido (—N═N+═N—), formyl (—(CO)—H), thioformyl (—(CS)—H), amino (—NH₂), mono- and di-(C1-C24 alkyl)-substituted amino, mono- and di-(C5-C20 aryl)-substituted amino, C2-C24 alkylamido (—NH—(CO)-alkyl), C5-C20 arylamido (—NH—(CO)-aryl), imino (—CR═NH where R=hydrogen, C1-C24 alkyl, C5-C20 aryl, C6-C20 alkaryl, C6-C20 aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O—), C1-C24 alkylsulfanyl (—S-alkyl; also termed “alkylthio”), arylsulfanyl (—S-aryl; also termed “arylthio”), C1-C24 alkylsulfinyl (—(SO)-alkyl), C5-C20 arylsulfinyl (—(SO)-aryl), C1-C24 alkylsulfonyl (—SO₂-alkyl), C5-C20 arylsulfonyl (—SO₂-aryl), phosphono (—P(O)(OH)₂), phosphonato (—P(O)(O⁻)₂), phosphinato (—P(O)(O⁻)), phospho (—PO₂), and phosphino (—PH₂), mono- and di-(C1-C24 alkyl)-substituted phosphino, mono- and di-(C5-C20 aryl)-substituted phosphine. In addition, the aforementioned functional groups may, if a particular group permits, be further substituted with one or more additional functional groups or with one or more hydrocarbyl moieties such as those specifically enumerated above.

When the term “substituted” appears prior to a list of possible substituted groups, it is intended that the term apply to every member of that group. For example, the phrase “substituted alkyl and aryl” is to be interpreted as “substituted alkyl and substituted aryl.”

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, =NR⁷⁰, ═N—OR⁷⁰, ═N₂ or =S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of 0, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl, N-morpholinyl, —N(H)₀₋₁(CH₂CH₂OH)₁₋₂, —NHCH₂CH₂N(CH₃)₂₋₃, —NHCH₂CH₂SO₃H, —NHCH₂CH₂PO₃H₂ and —NHCH₂CH₂CO₂H.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃ M⁺, —SO₃R⁷⁰, —OSO₂R⁷⁰, —OSO₃-M⁺, —OSO₃R⁷⁰, —PO₃₋₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

Salts include but are not limited to: Na, K, Ca, Mg, ammonium, tetraalkyl ammonium, aryl and alkyl sulfonates, phosphates, carboxylates, sulfates, Cl, Br, and guanidinium.

Unless otherwise specified, reference to an atom is meant to include isotopes of that atom. For example, reference to H is meant to include ¹H, ²H (i.e., D) and ³H (i.e., T), and reference to C is meant to include ¹²C and all isotopes of carbon (such as ¹³C).

In addition to the disclosure herein, in a certain embodiment, a group that is substituted has 1, 2, 3, or 4 substituents, 1, 2, or 3 substituents, 1 or 2 substituents, or 1 substituent.

Unless indicated otherwise, the nomenclature of substituents that are not explicitly defined herein are arrived at by naming the terminal portion of the functionality followed by the adjacent functionality toward the point of attachment. For example, the substituent “heterocycloalkyl(alkyl)” refers to the group (heterocycloalkyl)-(alkyl)-.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

In certain embodiments, a substituent may contribute to optical isomerism and/or stereo isomerism of a compound. Salts, solvates, hydrates, and prodrug forms of a compound are also of interest. Polymorphic, pseudo-polymorphic, amorphous and co-crystal forms of a compound are also of interest. All such forms are embraced by the present disclosure. Thus, the compounds described herein include salts, solvates, hydrates, prodrug and isomer forms thereof, including the pharmaceutically acceptable salts, solvates, hydrates, prodrugs and isomers thereof. In certain embodiments, a compound may be a metabolized into a pharmaceutically active derivative.

A “prodrug” is a derivative of a compound described herein, the pharmacologic action of which results from the conversion by chemical or metabolic processes in vivo to the active compound, including through release by cleavage of a linker. Prodrugs include compounds wherein an amino acid residue, or a polypeptide chain of two or more (e.g., two, three or four) amino acid residues is covalently joined through an amide or ester bond to a free amino, hydroxyl or carboxylic acid group of the compound. Additional types of prodrugs are also encompassed. For instance, free carboxyl groups can be derivatized as amides or alkyl esters. Prodrug esters as employed herein includes esters and carbonates formed by reacting one or more hydroxyls of compounds of the method of the invention with alkyl, alkoxy, or aryl substituted acylating agents employing procedures known to those skilled in the art to generate acetates, pivalates, methylcarbonates, benzoates and the like. As further examples, free hydroxyl groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxyl and amino groups are also included, as are carbonate prodrugs, sulfonate prodrugs, sulfonate esters and sulfate esters of hydroxyl groups. Free amines can also be derivatized to amides, sulfonamides or phosphonamides. All of the stated prodrug moieties may incorporate groups including but not limited to ether, amine and carboxylic acid functionalities. Moreover, any compound that can be converted in vivo to provide the bioactive agent (e.g., a compound of formula I) is a prodrug within the scope of the invention. Various forms of prodrugs are well known in the art. A comprehensive description of prodrugs and prodrug derivatives are described in: (a) The Practice of Medicinal Chemistry, Camille G. Wermuth et al., (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds., (Harwood Academic Publishers, 1991).

As used herein, the term “cargo”, “cargo moiety” or “molecular cargo” refers to any compound that is of interest for delivery to a cell. Cargo moieties of interest include, without limitation, small molecule drugs including without limitation antibiotics, antiviral agents, chemotherapeutic agents, nucleosides, polynucleotides, e.g. siRNA, mRNA, DNA, etc., proteins, fluorescent/radioactive/optical imaging agents, peptides/proteins/enzymes, nucleic acids (siRNA/RNA/DNA/etc.), metal based compounds/catalysts, polymers, site-specific cellular targeting agents (compounds/ligands/antibodies/etc.), for diverse applications such as treating infection, chemotherapeutic agents, smart adjuvants, gene therapy vectors, biosensors, bioreactors, and so forth. Any of a number of drugs are suitable for use as a cargo moiety, or can be modified to be rendered suitable for use in the subject compounds.

Cargo moieties of interest include, but are not limited to: antibiotics, e.g. antibiotics with the classes of aminoglycosides; carbapenems; and the like; penicillins, e.g. penicillin G, penicillin V, methicillin, oxacillin, carbenicillin, nafcillin, ampicillin, etc. penicillins in combination with β-lactamase inhibitors, cephalosporins, e.g. cefaclor, cefazolin, cefuroxime, moxalactam, etc; tetracyclines; cephalosporins; quinolones; lincomycins; macrolides; sulfonamides; glycopeptides including the anti-infective antibiotics vancomycin, teicoplanin, telavancin, ramoplanin and decaplanin. Derivatives of vancomycin include, for example, oritavancin and dalbavancin (both lipoglycopeptides). Telavancin is a semi-synthetic lipoglycopeptide derivative of vancomycin (approved by FDA in 2009). Other vancomycin analogs are disclosed, for example, in WO 2015022335 A1 and Chen et al. (2003) PNAS 100(10): 5658-5663, each herein specifically incorporated by reference. Non-limiting examples of antibiotics include vancomycin, linezolid, azithromycin, daptomycin, colistin, eperezolid, fusidic acid, rifampicin, tetracyclin, fidaxomicin, clindamycin, lincomycin, rifalazil, and clarithromycin.

Cargo moieties of interest include chemotherapeutic agents. Classes of chemotherapy drugs include alkylating agents; e.g. mechlorethamine, cyclophosphamide, chlorambucil, melphalan, ifosfamide; ethylenimines, eg. thiotepa, hexamethylmelamine; alkylsulfonates, e.g. busulfan; hydrazines and triazines, e.g. altretamine, procarbazine, dacarbazine, temozolomide; nitrosureas, e.g. carmustine, lomustine, streptozocin. Metal salts, e.g. carboplatin, cisplatin, oxaliplatin. Plant alkaloids include vinca alkaloids, e.g. vincristine, vinblastine, vinorelbine; taxanes, e.g. paclitaxel and docetaxel; podophyllotoxins, e.g. etoposide, tenisopide; camptothecan analogs, e.g. irinotecan, topotecan. Antitumor antibiotics include, for example, anthracyclines, e.g. doxorubicin, daunorubicin, epirubicin, mitoxantrone, idarubicin; chromomycins, e.g. dactinomycin and plicamycin; mitomycin and bleomycin. Antimetabolites include folic acid antagonist, e.g. methotrexate; pyrimidine antagonist, e.g. 5-fluorouracil, foxuridine, cytarabine, capecitabine, and gemcitabine; purine antagonist, e.g. 6-mercaptopurine and 6-thioguanine; adenosine deaminase inhibitor, e.g. cladribine, fludarabine, nelarabine and pentostatin. Topoisomerase inhibitors include topoisomerase I inhibitors, e.g. ironotecan, topotecan; topoisomerase II inhibitors, e.g. amsacrine, etoposide, etoposide phosphate, teniposide. Also included are ribonucleotide reductase inhibitor, e.g. hydroxyurea; adrenocortical steroid inhibitor, e.g. mitotane; enzymes, e.g. asparaginase and pegaspargase; antimicrotubule agent, e.g. estramustin; and retinoids, e.g. bexarotene, isotretinoin, tretinoin (atra). For example, specific cargos of interest include paclitaxel, doxorubicin, cisplatin, and bryostatin, etc. In some embodiments, the cargo moieties are anthracycline chemotherapeutic compounds, such as doxorubicin (DOX). Suitable cancer chemotherapeutic agents also include dolastatin and active analogs and derivatives thereof; and auristatin and active analogs and derivatives thereof (e.g., Monomethyl auristatin D (MMAD), monomethyl auristatin E (MMAE), monomethyl auristatin F (MMAF), and the like). See, e.g., WO 96/33212, WO 96/14856, and U.S. Pat. No. 6,323,315. Suitable cancer chemotherapeutic agents also include maytansinoids and active analogs and derivatives thereof (see, e.g., EP 1391213; and Liu et al (1996) Proc. Natl. Acad. Sci. USA 93:8618-8623); duocarmycins and active analogs and derivatives thereof (e.g., including the synthetic analogues, KW-2189 and CB 1-TM1); and benzodiazepines and active analogs and derivatives thereof (e.g., pyrrolobenzodiazepine (PBD). Also included are allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like. Other anti-proliferative cytotoxic agents are navelbene, CPT-11, anastrazole, letrazole, capecitabine, reloxafine, cyclophosphamide, ifosamide, and droloxafine.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; Vitamin D₃ analogs known to induce a tolerogenic dendritic cell phenotype; and adrenocortical suppressants, e.g. aminoglutethimide; 17a-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation; therefore compounds that bind to the estrogen receptor are used to block this activity. Corticosteroids may inhibit T cell proliferation.

Other suitable chemotherapeutic agents include, but are not limited to, metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include, but are not limited to, immunosuppressants, e.g. mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

Taxanes are suitable for use. “Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose). Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

Cargo moieties of interest include peptides and polypeptides, such as pVI (adenovirus lytic domain), TAT (HIV lytic domain), ovalbumin, NS5A1-31 (Hep C viral membrane anchor), etc.; GFP, MOMP (chlamydia protein), and EGF/EGFR, antibodies, etc.; Metals and metal ions such as Gold, Silver, Nickel and Copper (bead or catalyst), etc.; and nucleic Acids, such as DNA, RNA, and siRNA for any convenient gene of interest.

### Imaging Cargo Moieties.

Imaging cargo moieties include without limitation radiography moieties, e.g. heavy metals and radiation emitting moieties, positron emitting moieties, magnetic resonance contrast moieties, and optically visible moieties, e.g., fluorescent or visible-spectrum dyes, visible particles, etc.

Among the most commonly used positron-emitting nuclides in PET are included ¹¹C, ¹³N, ¹⁵O, and ¹⁸F. Isotopes that decay by electron capture and/or yemission are used in SPECT, and include ¹²³I and ⁹⁹mTc.

Magnetic resonance contrast moieties include chelates of chromium(III), manganese(II), iron(II), nickel(II), copper(II), praseodymium(II), neodymium(II), samarium(III) and ytterbium(III) ion. Because of their very strong magnetic moment, the gadolinium(II), terbium(III), dysprosium(III), holmium(III), erbium(III), and iron(III) ions are especially preferred. Examples of such chelates, suitable for magnetic resonance spin imaging, are described in U.S. Pat. No. 5,733,522, incorporated fully herein by reference. Nuclear spin contrast chelates may be conjugated to the anti-TBT antibody moieties through a suitable chemical linker.

Optically visible moieties for use as imaging moieties include fluorescent dyes, or visible-spectrum dyes, visible particles, and other visible labeling moieties. Fluorescent dyes such as ALEXA dyes, fluorescein, coumarin, rhodamine, bodipy Texas red, and cyanine dyes, are useful when sufficient excitation energy can be provided to the site to be inspected visually. Endoscopic visualization procedures may be more compatible with the use of such labels. Acceptable dyes include FDA-approved food dyes and colors, which are non-toxic, although pharmaceutically acceptable dyes which have been approved for internal administration are preferred. In preferred embodiments, such dyes are encapsulated in carrier moieties, which are in turn conjugated to the anti-TBT antibody. Alternatively, visible particles, such as colloidal gold particles or latex particles, may be coupled to the anti-TBT antibody moiety via a suitable chemical linker.

### Specific Binding Domains.

In some embodiments cargo comprises a specific binding domain, which provides a targeting moiety, e.g. to link the TG2 substrate or inhibitor to a targeted polypeptide. As used herein the term “specifically binds” refers to the degree of selectivity or affinity for which one molecule binds to another. In the context of binding pairs (e.g. a ligand/receptor, antibody/antigen, antibody/ligand, antibody/receptor binding pairs) a first molecule of a binding pair is said to specifically bind to a second molecule of a binding pair when the first molecule of the binding pair does not bind in a significant amount to other components present in the sample. A first molecule of a binding pair is said to specifically bind to a second molecule of a binding pair when the first molecule of the binding pair when the affinity of the first molecule for the second molecule is at least two-fold greater, at least ten times greater, at least 20-times greater, or at least 100-times greater than the affinity of the first molecule for other components present in the sample. In a particular embodiment, where the first molecule of the binding pair is an antibody, the antibody specifically binds to the second molecule of the binding pair (e.g. a protein, antigen, ligand, or receptor) if the affinity of the antibody for the second molecule of the binding pair is greater than about 109 liters/mole, alternatively greater than about 10¹⁰ liters/mole, greater than about 10¹¹ liters/mole, greater than about 10¹² liters/mole as determined by, e.g., Scatchard analysis (Munsen, et al. 1980 Analyt. Biochem. 107:220-239). Specific binding may be assessed using techniques known in the art including but not limited to competition ELISA, BIACORE® assays and/or KINEXA® assays, conducted at room temperature.

Binding domains of interest include, for example, designed binding proteins, ligands, antibodies and related binding proteins, and the like. In some embodiments a binding domain is an antibody, antibody fragment, or variant thereof. The term “antibody” herein is used in the broadest sense and specifically covers monoclonal antibodies, polyclonal antibodies, monomers, dimers, multimers, heavy chain only antibodies, three chain antibodies, single chain Fv, single domain antibodies, NANOBODIES®, etc., and also include antibody fragments with or without pegylation, so long as they exhibit the desired biological activity (Miller et al (2003) Jour. of Immunology 170:4854-4861).

In some embodiments a binding domain is an antibody binding moiety, and the like. In some embodiments an Ab binding moiety is a small molecule IgG binding group, for example as disclosed by Gong et al. (2016) Bioconjugate Chem. 2016, 27 (7), 1569-1573.

The term antibody may reference a full-length heavy chain, a full length light chain, an intact immunoglobulin molecule including a functional Fc sequence; or an immunologically active portion of any of these polypeptides, i.e., a polypeptide that comprises an antigen binding site that immunospecifically binds an antigen of a target of interest or part thereof.

The term “hypervariable region” when used herein refers to the amino acid residues of an antibody which are responsible for antigen-binding. The hypervariable region may comprise amino acid residues from a “complementarity determining region” or “CDR”, and/or those residues from a “hypervariable loop”. “Framework Region” or “FR” residues are those variable domain residues other than the hypervariable region residues as herein defined.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations, which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules; nanobodies or domain antibodies comprising single Ig domains from human or non-human species or other specific single-domain binding modules including non-antibody binding proteins such as, but not limited to, adnectins and anticalins; and multispecific or multivalent structures formed from antibody fragments.

The term “NANOBODY®” as used herein refers to a single domain antibody consisting of a single monomeric variable domain (also referred to as a variable heavy homodimer [V_{HH}]domain or immunoglobulin single variable domains or ISVs). The single domain antibodies are naturally produced by animals belonging to the camelid family. Nanobodies are smaller than human antibodies, where ISV are generally 12-15 kDa, human antibodies are generally 150-160 kDa, Fab fragments are ~50 kDa and single-chain variable fragments are ~25 kDa. NANOBODIES® provide specific advantages over traditional antibodies including smaller sizes, they are more easily engineered, higher chemical and thermo stability, better solubility, deeper tissue penetration, the ability to bind small cavities and difficult to access epitopes of target proteins, the ability to manufacture in microbial cells (i.e. cheaper production costs relative to animal immunization), and the like.

Unless indicated otherwise, the term “immunoglobulin single variable domain” or “ISV” is used as a general term to include but not limited to antigen-binding domains or fragments such as V_{HH} domains or V_{H} or V_{L} domains, respectively. The terms antigen-binding molecules or antigen-binding protein are used interchangeably and include also the term NANOBODIES®. The immunoglobulin single variable domains can be light chain variable domain sequences [e.g., a V_{L}-sequence), or heavy chain variable domain sequences (e.g., a V_{H}-sequence); more specifically, they can be heavy chain variable domain sequences that are derived from a conventional four-chain antibody or heavy chain variable domain sequences that are derived from a heavy chain antibody. Accordingly, the immunoglobulin single variable domains can be single domain antibodies, or immunoglobulin sequences that are suitable for use as single domain antibodies, “dAbs”, or immunoglobulin sequences that are suitable for use as dAbs, or NANOBODIES©, including but not limited to V_{HH} sequences. An amino acid sequence such as e.g. an immunoglobulin single variable domain or polypeptide is said to be a “VHH1 type immunoglobulin single variable domain” or “VHH type 1 sequence”, if said VHH1 type immunoglobulin single variable domain or VHH type 1 sequence has 85% identity (using the VHH1 consensus sequence as the query sequence and use the blast algorithm with standard setting, i.e., blosom62 scoring matrix) to the VHH1 consensus sequence and mandatorily has a cysteine in position 50, i.e., C50 (using Kabat numbering). See, for example, V_{HH} domains from Camelids in the article of Riechmann and Muyldermans, J. Immunol. Methods 2000 Jun. 23; 240 (1-2): 185-195.

The invention includes immunoglobulin sequences of different origin, comprising mouse, rat, rabbit, donkey, human and camelid immunoglobulin sequences. The immunoglobulin single variable domain includes fully human, humanized, otherwise sequence optimized or chimeric immunoglobulin sequences. An immunoglobulin variable domain and structure of an immunoglobulin single variable domain can be considered—without however being limited thereto—to be comprised of four framework regions or “FR's”, which are referred to in the art and herein as “Framework region 1” or “FR1”; as “Framework region 2” or “FR2”; as “Framework region 3” or “FR3; and as “Framework region 4” or “FR4”, respectively; which framework regions are interrupted by three complementary determining regions or “CDR's”, which are referred to in the art as “Complementarity Determining Region 1” or “CDR1”; as “Complementarity Determining Region 2” or “CDR2”; and as “Complementarity Determining Region 3” or “CDR3, respectively.

Such immunoglobulin single variable domains may be derived in any suitable manner and from any suitable source, and may for example be naturally occurring V_{HH} sequences (i.e., from a suitable species of Camelid, e.g., llama) or synthetic or semi-synthetic VHs or VLs (e.g., from human). Such immunoglobulin single variable domains may include “humanized” or otherwise “sequence optimized” VHHs, “camelized” immunoglobulin sequences (and in particular camelized heavy chain variable domain sequences, i.e., camelized VHs), as well as human VHs, human VLs, camelid VH Hs that have been altered by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing as further described herein.

### Linker.

The TG2 substrate/inhibitor and molecular cargo may be separated by a linker, e.g. a polypeptide linker, or a non-peptidic linker, etc. In some embodiments the linker is a rigid linker, in other embodiments the linker is a flexible linker. In some embodiments, the linker moiety is a peptide linker. In some embodiments, a peptide linker comprises 1 to 10 amino acids. In some embodiments, the peptide linker comprises 2, 3, 4, 5, 6, 7, 8, 9, 10 amino acids. Exemplary linkers include linear peptides having at least two amino acid residues such as Gly-Gly, Gly-Ala-Gly, Gly-Pro-Ala, Gly-Gly-Gly-Gly-Ser. Suitable linear peptides include poly glycine, polyserine, polyproline, polyalanine and oligopeptides consisting of alanyl and/or serinyl and/or prolinyl and/or glycyl amino acid residues. In one embodiment a linker comprises the amino acid sequence GSTSGSGKSSEGKG, or (GGGGS)n, where n is 1, 2, 3, 4, 5, etc.; however many such linkers are known and used in the art and may serve this purpose.

A “cleavable linker” is a linker that has one or more cleavable groups that may be broken by the result of a reaction or condition. The term “cleavable group” refers to a moiety that allows for release of a component of the solid support or oligomer of the invention by cleaving a bond linking the released moiety to the remainder of the conjugate. Exemplary cleavage mechanisms of use both in preparing and using the oligomers and solid supports of the invention are enzymatically or otherwise chemically mediated.

In addition to enzymatically cleavable groups, it is within the scope of the present invention to include one or more sites that are cleaved by the action of an agent other than an enzyme. Exemplary non-enzymatic cleavage agents include, but are not limited to, acids, bases, light (e.g., nitrobenzyl derivatives, phenacyl groups, ortho-hydroxcinnamate esters, benzoin esters), and heat. Many cleaveable groups are known in the art. See, for example, Jung et al., Biochem. Biophys. Acta, 761:152-162 (1983); Joshi et al., J. Biol. Chem., 265:14518-14525 (1990); Zarling et al., J. Immunol., 124: 913-920 (1980); Bouizar et al., Eur. J. Biochem., 155: 141-147 (1986); Park et al., J. Biol. Chem., 261: 205-210 (1986); Browning et al., J. Immunol., 143: 1859-1867 (1989). Moreover a broad range of cleavable, bifunctional (both homo- and hetero-bifunctional) spacer arms are commercially available.

An exemplary cleavable group is cleavable by a reagent, e.g. sodium hydroxide, ammonia or other amine. In various embodiments the cleavable linker is readily cleaved at room temperature or under heat. An example of linker used is a valine-citrulline dipeptide linker that is cleaved by cathepsin B in the lysosome.

Chemical groups that find use in linking binding domains include carbamate; amide (amine plus carboxylic acid); ester (alcohol plus carboxylic acid), thioether (haloalkane plus sulfhydryl; maleimide plus sulfhydryl), Schiff's base (amine plus aldehyde), urea (amine plus isocyanate), thiourea (amine plus isothiocyanate), sulfonamide (amine plus sulfonyl chloride), disulfide; hyrodrazone, lipids, and the like, as known in the art.

The linkage between binding domains may comprise spacers, e.g. alkyl spacers, which may be linear or branched, usually linear, and may include one or more unsaturated bonds; usually having from one to about 300 carbon atoms; more usually from about one to 25 carbon atoms; and may be from about three to 12 carbon atoms. Spacers of this type may also comprise heteroatoms or functional groups, including amines, ethers, phosphodiesters, and the like. Specific structures of interest include: (CH₂CH₂O)n where n is from 1 to about 12; (CH₂CH₂NH)n, where n is from 1 to about 12; [(CH₂)n(C═O)NH(CH₂)ₘ]_{z}, where n and m are from 1 to about 6, and z is from 1 to about 10; [(CH₂)nOPO₃(CH₂)ₘ]_{z} where n and m are from 1 to about 6, and z is from 1 to about 10. Such linkers may include polyethylene glycol, which may be linear or branched.

The TG2 substrate/inhibitor may comprise a homo- or heterobifunctional linker having a group at one end capable of forming a stable linkage to cargo. Illustrative entities include: azidobenzoyl hydrazide, N-[4-(p-azidosalicylamino)butyl]-3′-[2′-pyridyldithio]propionamide), bis-sulfosuccinimidyl suberate, dimethyladipimidate, disuccinimidyltartrate, N-γ-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, NHS-PEG-MAL; succinimidyl 4-[N-maleimidomethyl]cyclohexane-1-carboxylate; 3-(2-pyridyldithio)propionic acid N-hydroxysuccinimide ester (SPDP); N, N′-(1,3-phenylene) bismaleimide; N, N′-ethylene-bis-(iodoacetamide); or 4-(N-maleimidomethyl)-cyclohexane-1-carboxylic acid N-hydroxysuccinimide ester (SMCC); m-maleimidobenzoyl-N-hydroxysuccinimide ester (MBS), and succinimide 4-(p-maleimidophenyl)butyrate (SMPB), an extended chain analog of MBS. The succinimidyl group of these cross-linkers reacts with a primary amine, and the thiol-reactive maleimide forms a covalent bond with the thiol of a cysteine residue.

Other reagents useful for this purpose include: p,p′-difluoro-m,m′-dinitrodiphenylsulfone (which forms irreversible cross-linkages with amino and phenolic groups); dimethyl adipimidate (which is specific for amino groups); phenol-1,4-disulfonylchloride (which reacts principally with amino groups); hexamethylenediisocyanate or diisothiocyanate, or azophenyl-p-diisocyanate (which reacts principally with amino groups); disdiazobenzidine (which reacts primarily with tyrosine and histidine); O-benzotriazolyloxy tetramethuluronium hexafluorophosphate (HATU), dicyclohexyl carbodiimde, bromo-tris (pyrrolidino) phosphonium bromide (PyBroP); N,N-dimethylamino pyridine (DMAP); 4-pyrrolidino pyridine; N-hydroxy benzotriazole; and the like. Homobifunctional cross-linking reagents include bismaleimidohexane (“BMH”).

“Small molecule drug” as used herein refers to a compound, e.g., an organic compound, naturally occurring or non-naturally occurring, which exhibits a pharmaceutical activity of interest and which is generally of a molecular weight of 800 Da or less, or 2000 Da or less, but can encompass molecules of up to 5 kDa and can be as large as 10 kDa. A small inorganic molecule refers to a molecule containing no carbon atoms, while a small organic molecule refers to a compound containing at least one carbon atom. “Peptide drug” as used herein refers to amino-acid containing polymeric compounds, and is meant to encompass naturally-occurring and non-naturally-occurring peptides, oligopeptides, cyclic peptides, polypeptides, and proteins, as well as peptide mimetics. The peptide drugs may be obtained by chemical synthesis or be produced from a genetically encoded source (e.g., recombinant source). Peptide drugs can range in molecular weight, and can be from 200 Da to 10 kDa or greater.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and native leader sequences, with or without N-terminal methionine residues; immunologically tagged proteins; fusion proteins with detectable fusion partners, e.g., fusion proteins including as a fusion partner a fluorescent protein, p-galactosidase, luciferase, etc.; and the like.

The terms “nucleic acid molecule”, “oligonucleotide” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), small interfering RNA (siRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

A “therapeutically effective amount” or “efficacious amount” means the amount of a compound that, when administered to a mammal or other subject for treating a disease, condition, or disorder, is sufficient to effect such treatment for the disease, condition, or disorder. The “therapeutically effective amount” will vary depending on the compound, the disease and its severity and the age, weight, etc., of the subject to be treated.

The term “unit dosage form,” as used herein, refers to physically discrete units suitable as unitary dosages for human and animal subjects, each unit containing a predetermined quantity of a compound (e.g., an aminopyrimidine compound, as described herein) calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

The terms “subject,” “individual,” and “patient” are used interchangeably herein to refer to a mammal being assessed for treatment and/or being treated. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc.

The term “sample” with reference to a patient encompasses blood and other liquid samples of biological origin, solid tissue samples such as a biopsy specimen or tissue cultures or cells derived therefrom and the progeny thereof. The term also encompasses samples that have been manipulated in any way after their procurement, such as by treatment with reagents; washed; or enrichment for certain cell populations, such as diseased cells. The definition also includes samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's diseased cell, e.g., a sample comprising polynucleotides and/or polypeptides that is obtained from a patient's diseased cell (e.g., a cell lysate or other cell extract comprising polynucleotides and/or polypeptides); and a sample comprising diseased cells from a patient. A biological sample comprising a diseased cell from a patient can also include non-diseased cells.

“In combination with”, “combination therapy” and “combination products” refer, in certain embodiments, to the concurrent administration to a patient of the engineered proteins and cells described herein in combination with additional therapies, e.g. surgery, radiation, chemotherapy, and the like. When administered in combination, each component can be administered at the same time or sequentially in any order at different points in time. Thus, each component can be administered separately but sufficiently closely in time so as to provide the desired therapeutic effect.

“Concomitant administration” means administration of one or more components, such as engineered proteins and cells, known therapeutic agents, etc. at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

### Compositions

A TG2 substrate/inhibitor of the disclosure comprises or consists of a polypeptide comprising the pentapeptide sequence Pro-X-Y-Pro-R (formula I), where X is selected from amino acids that engage TG2 through the formation of a covalent enzyme-compound intermediate, including without limitation glutamine, a-diazoketones, a-halo-ketones, up-unsaturated carbonyl compounds, and ab-unsaturated sulfones. In some embodiments X is glutamine. Y is any amino acid, including unnatural amino acids with easily appended functional groups, including without limitation primary/secondary amines, alcohols, and carboxylic acids). R is a natural or non-natural aromatic amino acid, e.g. tyrosine (Y), phenylalanine (F), and tryptophan (W), naphthalene, etc. The C-terminus of the pentapeptide can be a carboxylate, an ester, or an amide. Molecular cargo or a linker is linked to the C-terminus of the pentapeptide, and/or to Y. The cargo may be joined to the pentapeptide through a linker, through covalent direct conjugation, through non-covalent high affinity pairing, and the like. In some embodiments a TG2 substrate/inhibitor comprises a linker suitable for joining to cargo.

Molecular cargo include any compound that is of interest for delivery to a cell. Cargo moieties of interest include, without limitation, antibiotics, antiviral agents, chemotherapeutic agents, nucleosides, polynucleotides, e.g. siRNA, mRNA, DNA, etc., proteins, fluorescent/radioactive/optical imaging agents, peptides/proteins/enzymes, nucleic acids (siRNA/RNA/DNA/etc.), metal based compounds/catalysts, polymers, site-specific cellular targeting agents (compounds/ligands/antibodies/etc.), immunomodulatory drugs, antigen binding moieties, etc., and are useful for diverse applications such as imaging, treating infection, chemotherapeutic agents, smart adjuvants, gene therapy vectors, biosensors, bioreactors, and so forth. Molecular cargo can be joined to the substrate/inhibitor through a linker, optionally a cleavable linker.

In one embodiment, the TG2 substrate/inhibitor comprises or consists of Formula II:

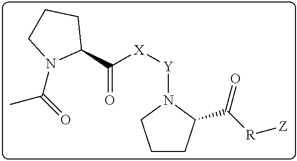

where X is selected from:

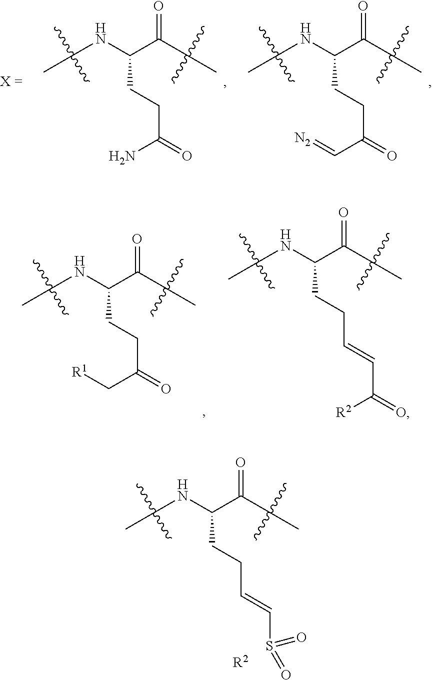

where R¹ is selected from Cl, Br, I, OSO₂CF₃; R² is selected from N(CH₃)₂, OCH₃, NH₂, CH₃, etc.;
Y is any amino acid,

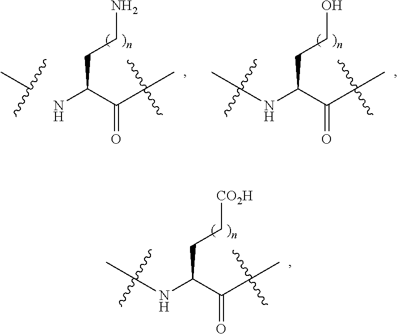

or molecular cargo, or a linker. In some embodiments Y is leu (L)
R is an aryl, heteroaryl or substituted aryl group. In some embodiments R is Ph, CH₂Ph, CH₂PhOH, CH₂Naphthelene.
   and
Z is OH, OR³, NH₂, NHR³, etc., or Z is molecular cargo or a linker. R³ is an alkyl, e.g. a lower alkyl, substituted alkyl, heteroalkyl, etc.

In some embodiments, an imaging composition is provided, comprising or consisting of a TG2 substrate/inhibitor of the disclosure, linked to an imaging moiety. Imaging moieties are as described herein, and include without limitation radiography moieties, e.g. heavy metals and radiation emitting moieties, positron emitting moieties, magnetic resonance contrast moieties, and optically visible moieties, e.g., fluorescent or visible-spectrum dyes, visible particles, etc. In some embodiments an imaging composition is AcPro-HWE-(Dab-SCy5)-Pro-Phe (“HB-230”), where HWE is the unnatural amino acid (S,E)-2-amino-7-(dimethylamino)-7-oxohept-5-enoic acid, Dab is the unnatural amino acid (S)-2,4-diaminobutanoic acid, and SCy5 is the small molecule fluorophore sulfo-cyanine 5.

A compound of interest is HB-230 or HB-2121, as shown below.

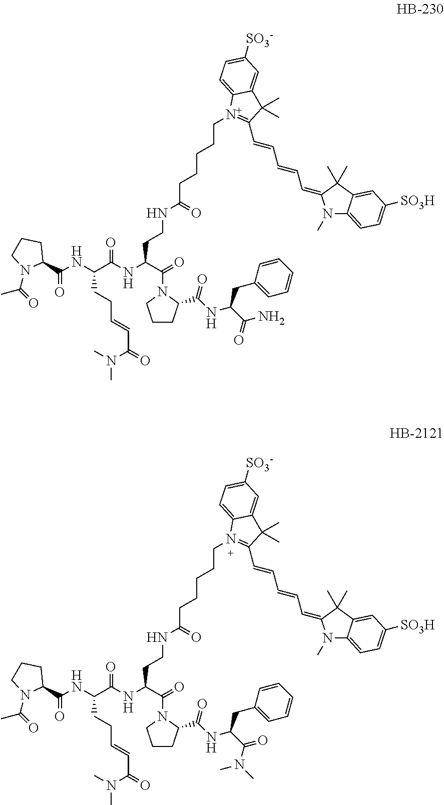

In some embodiments, a targeting composition is provided, comprising a TG2 substrate/inhibitor of the disclosure, linked to a targeting moiety, e.g. a binding domain specific for a targeted molecule. Specific binding domains are as described herein, and may comprise, for example, an antibody or fragment thereof that specifically binds to a targeted polypeptide. In some embodiments a targeted polypeptide is a pathogenically relevant extracellular protein.

A compound of interest specifically targeted to IgG proteins is HB-328:

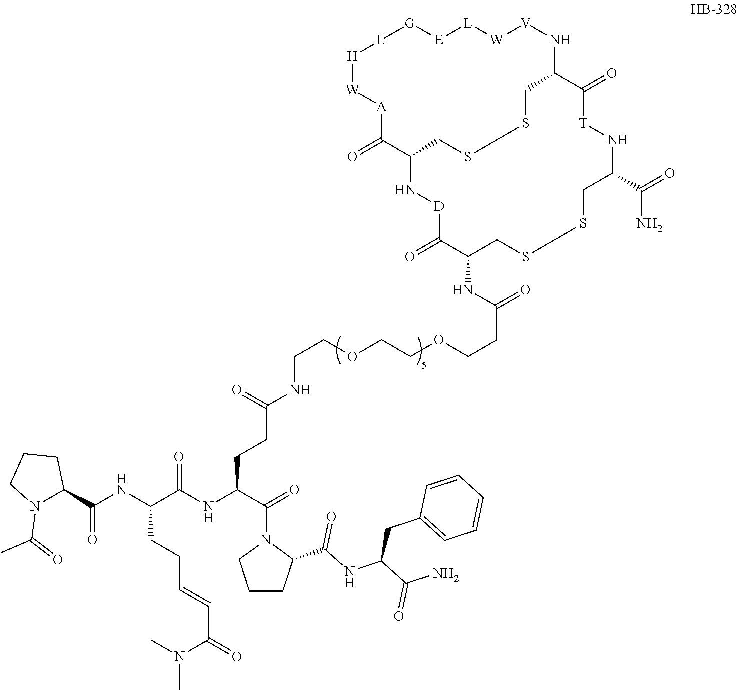

In an embodiment, a therapeutic composition is provided, comprising a TG2 substrate/inhibitor of the disclosure, linked to therapeutic moiety, e.g. molecular cargo comprising a small molecule drug, such as chemotherapeutic, antibiotic, anti-viral, etc. The cargo may be joined to the TG2 substrate/inhibitor through a linker. The linker may be a linker that is cleaved in the lysosomal complex, thereby releasing the cargo.

The substrate or inhibitor, optionally with a linker or cargo, may be provided in a pharmaceutical composition. The pharmaceutical composition may be provided in a unit dose. Dosage and frequency may vary depending on the half-life of the agent in the patient. It will be understood by one of skill in the art that such guidelines will be adjusted for the molecular weight of the agent, the clearance from the blood, the mode of administration, and other pharmacokinetic parameters. The dosage may also be varied for localized administration, e.g. intranasal, inhalation, etc., or for systemic administration, e.g. i.m., i.p., i.v., oral, and the like.

The TG2 substrate or inhibitor and cargo can be administered by any suitable means, including topical, oral, parenteral, intrapulmonary, and intranasal. Parenteral infusions include intramuscular, intravenous (bolus or slow drip), intraarterial, intraperitoneal, intrathecal or subcutaneous administration. An agent can be administered in any manner which is medically acceptable. This may include injections, by parenteral routes such as intravenous, intravascular, intraarterial, subcutaneous, intramuscular, intratumor, intraperitoneal, intraventricular, intraepidural, or others as well as oral, nasal, ophthalmic, rectal, or topical. Sustained release administration is also specifically included in the disclosure, by such means as depot injections or erodible implants.

As noted above, a TG2 substrate or inhibitor and cargo can be formulated with an a pharmaceutically acceptable carrier (one or more organic or inorganic ingredients, natural or synthetic, with which a subject agent is combined to facilitate its application). A suitable carrier includes sterile saline although other aqueous and non-aqueous isotonic sterile solutions and sterile suspensions known to be pharmaceutically acceptable are known to those of ordinary skill in the art. An “effective amount” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

A TG2 substrate or inhibitor and cargo can be administered as a pharmaceutical composition comprising a pharmaceutically acceptable excipient. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

In pharmaceutical dosage forms, the active agents and/or other compounds may be administered in the form of their pharmaceutically acceptable salts, or they may also be used alone or in appropriate association, as well as in combination with other pharmaceutically active compounds. The agents may be combined, as previously described, to provide a cocktail of activities. The following methods and excipients are exemplary and are not to be construed as limiting the invention.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

In one embodiment of the invention, the oral formulations comprise enteric coatings, so that the active agent is delivered to the intestinal tract. A number of methods are available in the art for the efficient delivery of enterically coated proteins into the small intestinal lumen. Most methods rely upon protein release as a result of the sudden rise of pH when food is released from the stomach into the duodenum, or upon the action of pancreatic proteases that are secreted into the duodenum when food enters the small intestine. For intestinal delivery of a PEP and/or a glutamine specific protease, the enzyme is usually lyophilized in the presence of appropriate buffers (e.g. phosphate, histidine, imidazole) and excipients (e.g. cryoprotectants such as sucrose, lactose, trehalose). Lyophilized enzyme cakes are blended with excipients, then filled into capsules, which are enterically coated with a polymeric coating that protects the protein from the acidic environment of the stomach, as well as from the action of pepsin in the stomach. Alternatively, protein microparticles can also be coated with a protective layer. Exemplary films are cellulose acetate phthalate, polyvinyl acetate phthalate, hydroxypropyl methylcellulose phthalate and hydroxypropyl methylcellulose acetate succinate, methacrylate copolymers, and cellulose acetate phthalate.

Other enteric formulations comprise engineered polymer microspheres made of biologically erodable polymers, which display strong adhesive interactions with gastrointestinal mucus and cellular linings and can traverse both the mucosal absorptive epithelium and the follicle-associated epithelium covering the lymphoid tissue of Peyer's patches. The polymers maintain contact with intestinal epithelium for extended periods of time and actually penetrate it, through and between cells. See, for example, Mathiowitz et al. (1997) Nature 386 (6623): 410-414. Drug delivery systems can also utilize a core of superporous hydrogels (SPH) and SPH composite (SPHC), as described by Dorkoosh et al. (2001) J Control Release 71(3):307-18.

Formulations are typically provided in a unit dosage form, where the term “unit dosage form,” refers to physically discrete units suitable as unitary dosages for human subjects, each unit containing a predetermined quantity of active agent in an amount calculated sufficient to produce the desired effect in association with a pharmaceutically acceptable diluent, carrier or vehicle. The specifications for the unit dosage forms of the present invention depend on the particular complex employed and the effect to be achieved, and the pharmacodynamics associated with each complex in the host.

The pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are commercially available. Any compound useful in the methods and compositions of the invention can be provided as a pharmaceutically acceptable base addition salt. “Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

In some embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

A carrier may bear the agents in a variety of ways, including covalent bonding either directly or via a linker group, and non-covalent associations. Suitable covalent-bond carriers include proteins such as albumins, peptides, and polysaccharides such as aminodextran, each of which have multiple sites for the attachment of moieties. The nature of the carrier can be either soluble or insoluble for purposes of the invention.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Compositions can be prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the active agents can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in further optimizing and/or defining a therapeutic dosage range and/or a sub-therapeutic dosage range (e.g., for use in humans). The exact formulation, route of administration and dosage can be chosen by the individual physician in view of the patient's condition.

### Methods of Use

In some embodiments, methods are provided for linking a molecular cargo of interest to a TG2 substrate/inhibitor, for delivery of the molecules to endo-lysosomal compartments of LRP1-expressing cells. In some embodiments, a TG2 substrate/inhibitor is provided, comprising a linker suitable for attaching a molecular cargo of interest. In some embodiments, a TG2 substrate/inhibitor is provided comprising an affinity reagent, e.g. biotin, for attaching avidin-conjugated cargo. In some embodiments a TG2 substrate/inhibitor is provided, comprising a suitable reactive group for conjugating to a molecular cargo of interest.

In some embodiments, methods provided for imaging in vivo, e.g., to locate or identify sites where cells or polypeptides, e.g. catalytically active TG2, are present. In these embodiments, an imaging composition as disclosed herein is administered to an individual (e.g., orally or by injection), and labeled cells are located using standard imaging techniques, including, but not limited to, magnetic resonance imaging, computed tomography scanning, and the like.

For diagnostic in vivo imaging, the type of detection instrument available is a major factor in selecting a given radionuclide. The radionuclide chosen must have a type of decay that is detectable by a given type of instrument. In general, any conventional method for visualizing diagnostic imaging can be utilized in accordance with this invention. Another important factor in selecting a radionuclide for in vivo diagnosis is that its half-life be long enough that it is still detectable at the time of maximum uptake by the target tissue, but short enough that deleterious radiation of the host is minimized.

The detectably labeled TG2 substrate/inhibitor is used in conjunction with imaging techniques, in order to analyze the presence of active TG2 protein. In one embodiment, the imaging method is one of PET or SPECT, which are imaging techniques in which a radionuclide is synthetically or locally administered to a patient. The subsequent uptake of the radiotracer is measured over time and used to obtain information about the targeted tissue. Because of the high-energy (γ-ray) emissions of the specific isotopes employed and the sensitivity and sophistication of the instruments used to detect them, the two-dimensional distribution of radioactivity may be inferred from outside of the body.

Typically the dosage will be 0.001 to 100 milligrams of conjugate per kilogram subject body weight. Doses in the range of 0.01 to 1 mg per kilogram of patient body weight may be utilized for a radionuclide therapeutic composition which is administered intrathecally. Relatively large doses, in the range of 0.1 to 10 mg per kilogram of patient body weight, may used for imaging conjugates with a relatively non-toxic imaging moiety. The amount utilized will depend on the sensitivity of the imaging method, and the relative toxicity of the imaging moiety.

The effective amount of a therapeutic or imaging composition to be given to a particular patient will depend on a variety of factors, several of which will be different from patient to patient. A competent clinician will be able to determine an effective amount of a therapeutic agent to administer to a patient, or an effective amount of an imaging composition to administer to a patient to facilitate visualization. Dosage will depend on the treatment, route of administration, the nature of the therapeutics, sensitivity to the therapeutics, etc. Utilizing LD₅₀ animal data, and other information available, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Imaging moieties are typically less toxic than cytotoxic moieties and may be administered in higher doses in some embodiments. Utilizing ordinary skill, the competent clinician will be able to optimize the dosage of a particular therapeutic or imaging composition in the course of routine clinical trials.

The compositions can be administered to the subject in a series of more than one administration. For therapeutic compositions, regular periodic administration (e.g., every 2-3 days) will sometimes be required, or may be desirable to reduce toxicity. For therapeutic compositions which will be utilized in repeated-dose regimens, antibody moieties which do not provoke immune responses are preferred. The imaging antibody conjugate compositions may be administered at an appropriate time before the visualization technique. For example, administration within an hour before direct visual inspection may be appropriate, or administration within twelve hours before an MRI scan may be appropriate. Care should be taken, however, to not allow too much time to pass between administration and visualization, as the imaging compound may eventually be cleared from the patient's system.

In other embodiments, methods are provided for the treatment of conditions such as cancer, infections, autoimmune disease, inflammation, and the like, by administering an effective dose of a therapeutic composition as disclosed herein, comprising a TG2 substrate/inhibitor as disclosed herein, linked to a therapeutic cargo moiety.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Design, Synthesis, and Analysis of HB-230, an Imaging Tool for Catalytically Active TG2:

The amino acid sequence of HB-225 (Scheme 1) is modeled after a preferred TG2 substrate, PQLPY, three copies of which are found in the aforementioned 33-residue gluten peptide. To generate an irreversible inhibitor of TG2, the reactive Gln residue of PQLPY was replaced with an isosteric unnatural amino acid harboring an α,β-unsaturated dimethylamide “soft” electrophile (Campbell et al.) The terminal Tyr (Y) residue was replaced with proteinogenic Phe (F) for synthetic convenience, as the phenolic substituent of Tyr was also not predicted to be relevant to TG2 or HLA-DQ2 recognition based on co-crystal structures of both proteins (Kim et al., 2004; Pinkas et al., 2007).

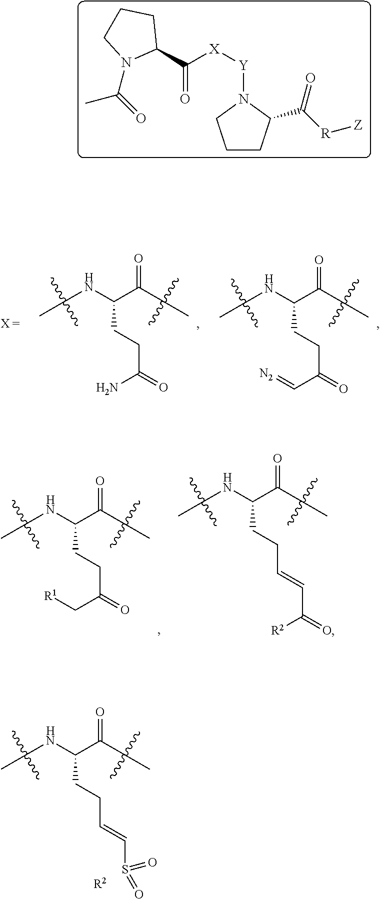

R¹═Cl, Br, I, OSO₂CF₃ etc.
R²═NMe₂, OMe, NH₂, Me, etc.
Y=any amino acid,

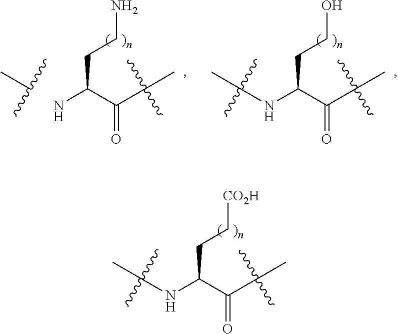

R=Aromatic amino acid (eg. phenylalanine, tyrosine, tryptophan, etc.)
Z═OH, OR³, NH₂, NHR³, etc.
R³=Me, Et, etc.

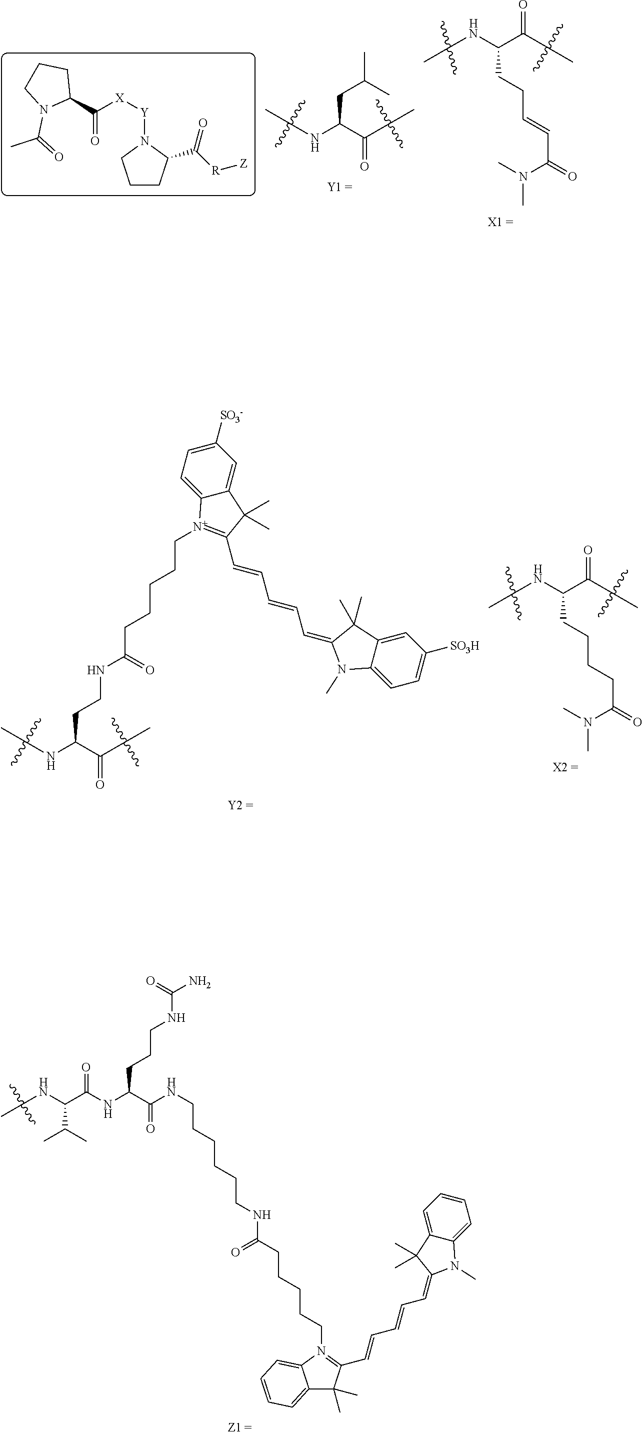

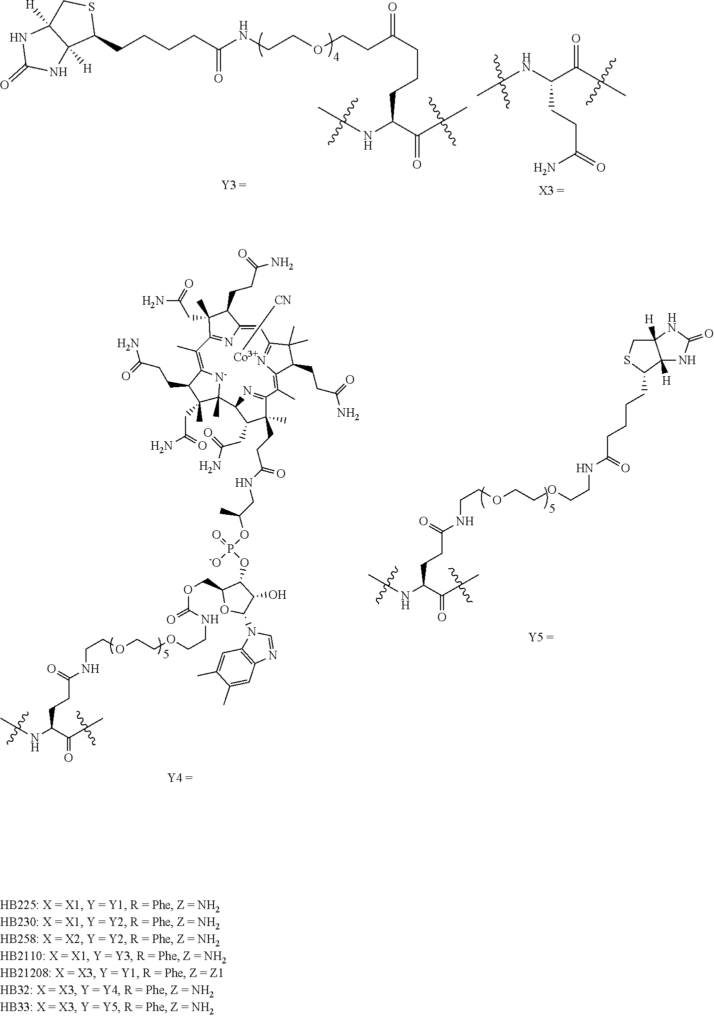

Scheme 1, specific structures of molecules in this disclosure.

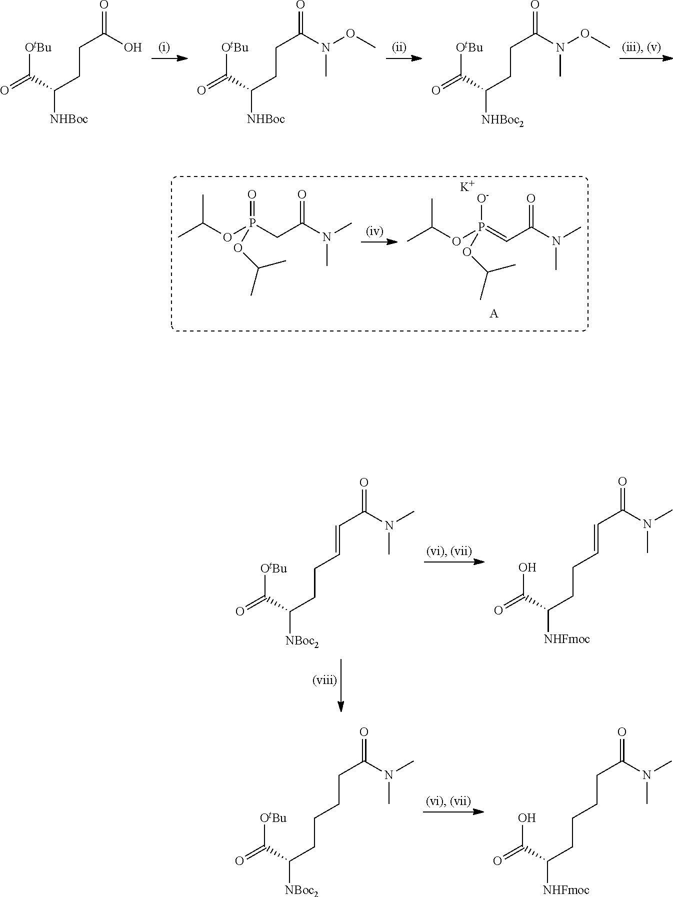

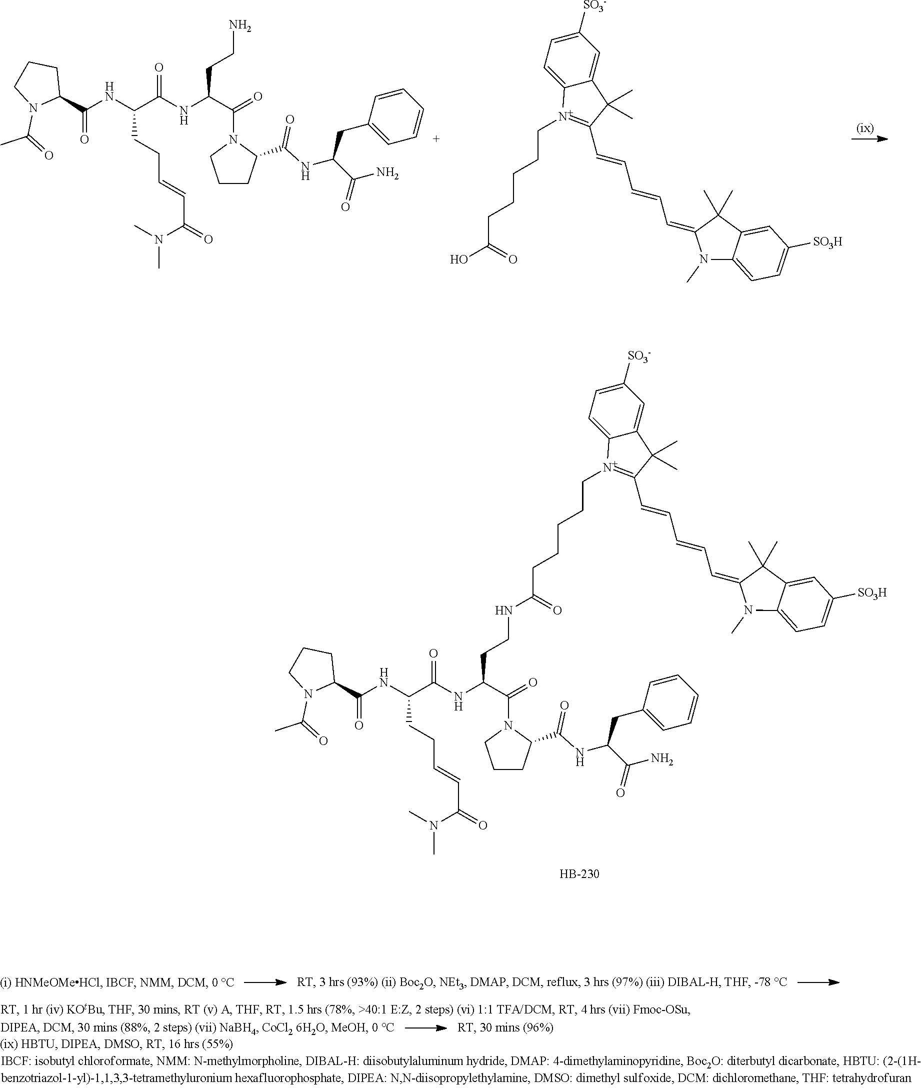

Scheme 2. Synthetic routes to key compounds. Peptides were synthesized by fmoc solid-phase peptide synthesis (only HB-230 is shown for clarity).

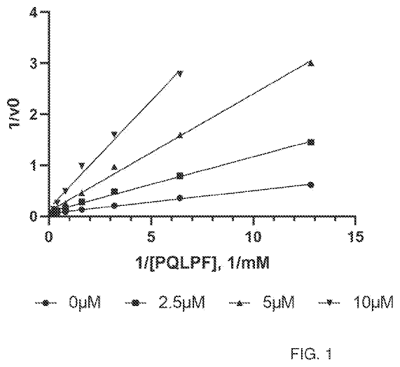

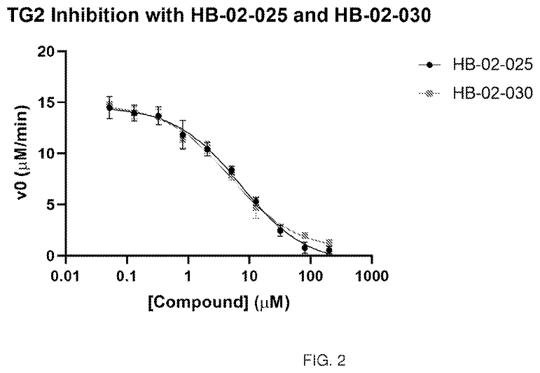

Steady-state kinetic analysis (FIG. 1) revealed that HB225 inactivated human TG2 with high specificity (kᵢ=0.05 min⁻¹, Kᵢ=13 μM). Encouraged by this finding, we went on to design and synthesize our target fluorescent probe, HB230, in which a second unnatural amino acid residue, diaminobutyrate (DAB), was installed in place of Leu (L) in HB225. This residue was also predicted to neither be recognized by TG2 nor HLA-DQ2. Attachment of a Sulfo-Cy5 fluorophore via amide linkage to the DAB residue yielded the desired probe. As anticipated, HB230 had comparable potency to HB225 (IC₅₀=4.9 μM versus 5.1 μM respectively, under equivalent assay conditions, FIG. 2).

### HB230 Induces α₂-Macroglobulin-Dependent Endocytosis of Catalytically Active TG2:

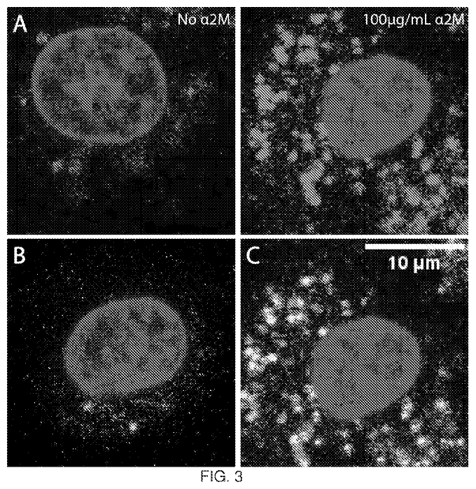

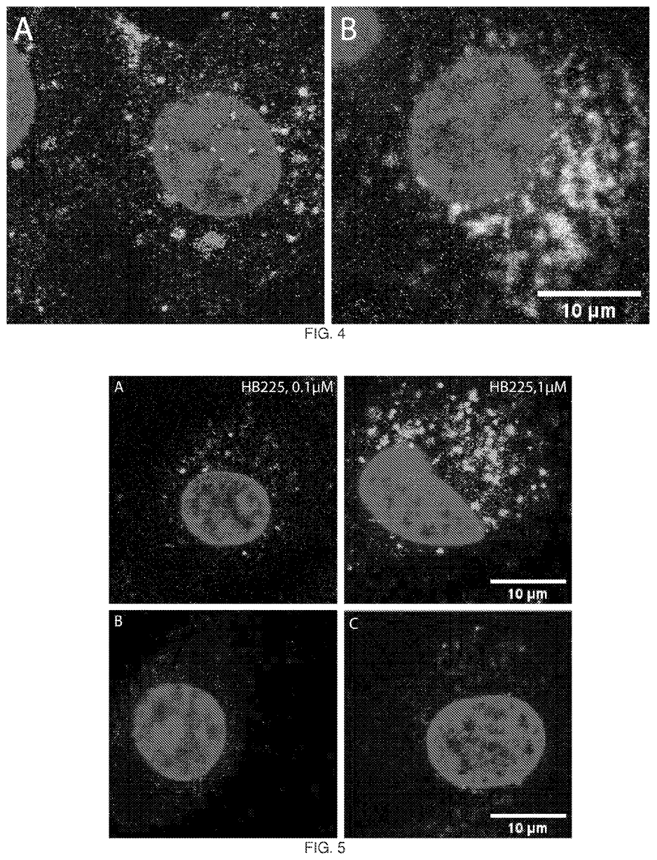

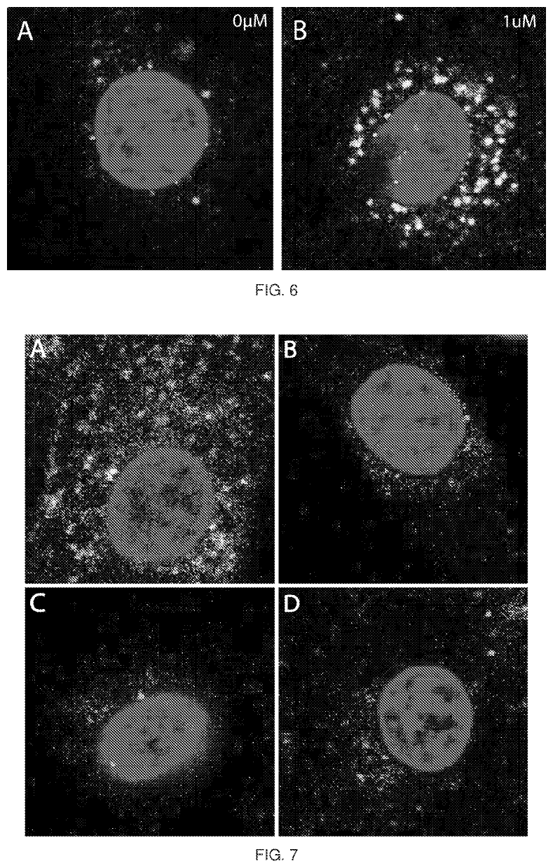

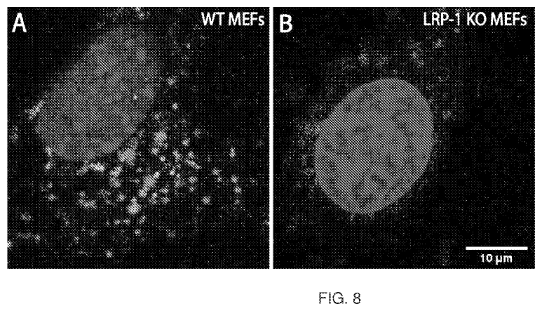

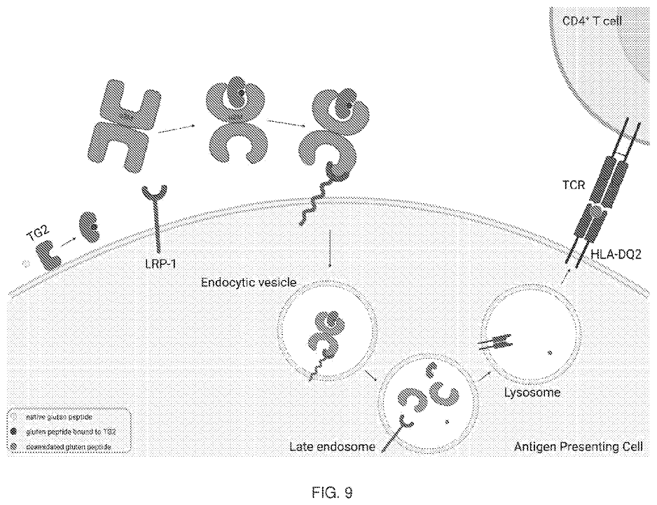

As seen in FIG. 3A, normal rat kidney (NRK) cells showed robust uptake of 1 TM exogenous HB230 in 90 min. Puncta of two distinct sizes were observed. Supplementation of NRK cell cultures with α2M led to a strong dose-dependent increase in uptake of HB230 up to ~100 μg/ml, indicative of a saturable mechanism. (Note that the physiological concentration of α₂M in human plasma typically exceeds 1 mg/mL (Ganrot and Scherstén, 1967).) The addition of α₂M predominantly appeared to affect the size and number of the larger puncta, which are indicative of receptor mediated endocytosis uptake.

To verify that HB230 internalization was dependent on TG2 activity, HB258 was designed and synthesized as a control probe. This dihydro-analog is otherwise identical to HB230 but lacks its electrophilic warhead and is therefore unable to irreversibly bond to the active site C277 residue of TG2. As seen in FIG. 3B, HB258 does not undergo significant uptake by NRK cells even at high concentrations of both the probe and α₂M in the culture medium. Notably, this fluorescent probe only appears as small intracellular puncta; the larger puncta formed by HB230 are absent.

To obtain definitive evidence for co-endocytosis of HB230 and α₂M, a fluorescent batch of α₂M was prepared. Using this probe, a strong overlap was observed between the fluorescent signals of HB230 (green) and α₂M (red) in the larger, but not smaller, puncta (FIG. 3C).

### Characterizing the Large Puncta Harboring Colocalized TG2 and α₂M:

To establish the identity of these large intracellular puncta, NRK cells were transfected with RFP tagged lysosomal-associated membrane protein (LAMP-1). Wild-type and transfected cells were co-incubated with α₂M and HB230 for 90 min prior to fixation. To test the hypothesis that the large puncta corresponded to early endosomes, fixed wild-type cells were stained with an antibody against early endosome antigen-1 (EEA-1). As observed in FIG. 4A, EEA-1 and HB230 appear in distinct sub-cellular compartments for the most part, implying that vesicles derived from internalization of the HB230/TG2/α₂M complex did not fuse with early endosomes. In contrast, HB230 strongly co-localizes with RFP-tagged LAMP-1 in the large puncta (FIG. 4B), implying that internalized vesicles harboring the ternary complex comprised of HB230, TG2 and α₂M are escorted to the lysosome.

### Conformational Requirements for TG2 Recognition by α₂M:

To identify the conformational requirements for high-affinity recognition of TG2 by α₂M, fluorescently labeled α₂M was added to NRK cell cultures in the presence of different (unlabeled) TG2 inhibitors. As shown in FIG. 5, HB225 promoted dose-dependent formation of larger α₂M-labeled puncta, whereas a different active site-targeted irreversible inhibitor, CK805 (Klöck et al., 2014), did not. Cystamine is a small molecule oxidant that inactivates TG2 by promoting C370-C371 disulfide bond formation even in the absence of active site occupancy (Palanski and Khosla, 2018); it too was unable to promote rapid internalization of fluorescent α₂M by itself (FIG. 5). Together, our data suggests that α₂M-promoted endocytosis can be achieved by some but not all forms of bound or unoccupied TG2.

### Endocytosis of Antigenic Gluten Peptides Via Ternary Complex Formation:

Thus far, endocytosis of the TG2-α₂M complex had exclusively been visualized using peptidic probes that irreversibly label the active site of TG2. To investigate whether transient enzyme-substrate complexes can be efficiently internalized in an α₂M-dependent manner, NRK cells were co-incubated with fluorescent α₂M and the aforementioned 33-residue gluten peptide (33 mer) tagged with a Sulfo-Cy5 fluorophore. An entirely analogous pattern of co-localized endocytosis was observed between these two fluorescent probes (FIG. 6) as between α₂M and HB230.

### LRP-1 is Responsible for Receptor-Mediated Endocytosis of Ternary Complexes:

Low density lipoprotein (LDL) receptor-related protein 1 (LRP-1, a.k.a., (α₂M receptor or CD91), a member of the LDL receptor family, is responsible for receptor-mediated endocytosis of α₂M. Although this receptor is expressed on the surface of a wide range of cell types within the body, α₂M endocytosis by LRP-1 is only triggered when this ubiquitous plasma protein undergoes a conformational change upon binding to its protease or non-protease ligands (Bres and Faissner, 2019). We sought to establish whether endocytosis of the ternary α₂M-TG2-peptide complex was mediated by LRP-1 using pharmacologic inhibitors of clathrin mediated endocytosis. In comparison to untreated NRK cells, cells treated with methyl-β-cyclodextrin (MβCD), Pitstop-2 or Dyno-4a showed strong inhibition of α₂M and peptide colocalization as large endocytic puncta (FIG. 7). To demonstrate the role of LRP-1 in this receptor-mediated endocytic process, WT MEFs were compared to LRP-1 knockout MEFs. Whereas wild-type MEFs also internalized the ternary complex generated by the addition of HB230 and 02M, knockout MEFs showed a marked defect in their ability to form large co-stained puncta under the same conditions (FIG. 8).

Based on all of the above data, the following sequence of events is proposed (FIG. 9): TG2 binds to a substrate at or near the cell surface; the resulting covalent complex is recognized by α₂M, leading to the formation of a ternary complex that undergoes LRP-1 mediated endocytosis. The deamidated product released upon internalization is trafficked into the lysosome.

### Specific Clearance of Target Proteins to the Lysosome Through the LRP-1 Pathway:

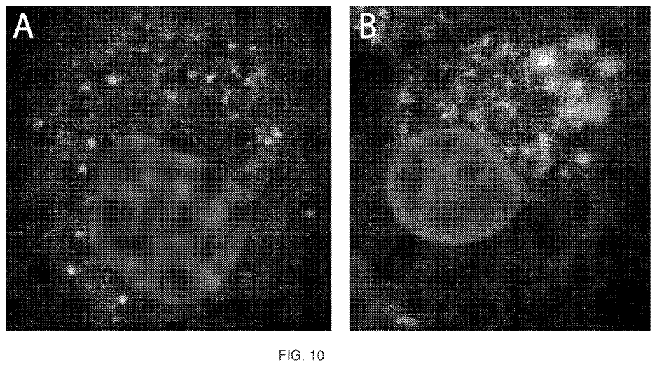

In order to determine the ability of this pathway to clear specifically targeted proteins from the extracellular environment, we synthesized a pentapeptide harboring the electrophilic warhead and a PEG-4 biotin conjugated to the DAB substituted Y amino acid (HB2110). Streptavidin is expected to bind to the biotin arm of the probe, and thus be endocytosed along with the rest of the complex as previously described. To test this hypothesis NRK cells were treated with 2 μM HB2110 and 1 μM Cy5-conjugated streptavidin. The Cy5-Streptavidin was found to colocalize with α₂M in the lysosomal compartment (FIG. 10) indicating that this mechanism can be used to clear proteins that have small molecule ligands from the extracellular environment.

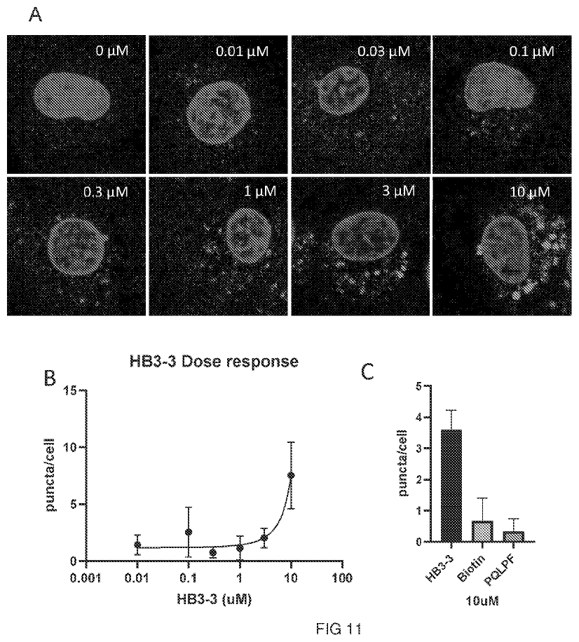

We also synthesized the equivalent pentapeptide harbouring a Gln residue in the place of the electrophilic warhead, with biotin conjugated to the DAB substituted Y amino acid through a PEG linker (HB3-3). We then assed the ability of cells to endocytose fluorescent streptavidin in the presence of HB3-3 and α₂M. In the presence of α₂M fluorescent streptavidin is endocytosed in an HB3-3 dose dependent manner (FIG. 11). Individual components of the bifunctional molecule do not promote endocytosis.

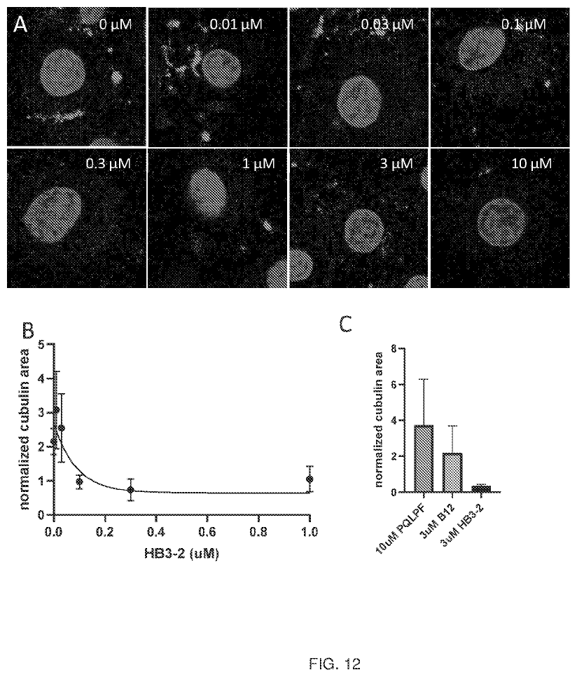

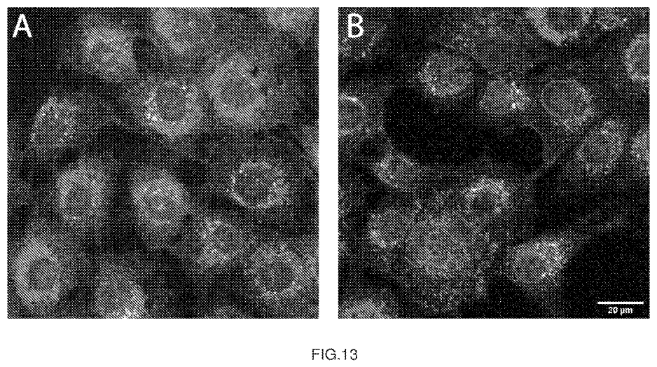

Cell surface proteins can also be pharmacological targets. Thus we targeted cubilin, the canonical receptor for the intrinsic factor (IF)-vitamin B₁₂ complex. We synthesized a pentapeptide harbouring a Gln residue in the X position, and vitamin B₁₂ conjugated to the DAB substituted Y amino acid through a PEG-5 linker (HB3-2). In the presence of 30 μg/mL intrinsic factor, cell surface cubilin is depleted in an HB3-2 dependent manner, as seen in FIG. 12. Cubilin is stained using a sheep anti cubilin antibody which recognizes rat cubilin and an ovine Alexa-647 secondary antibody. Inhibiting LRP-1 through RAP mitigates the depletion of surface cubilin. Individual components alone (vitamin B₁₂ and PQLPF) do not promote the depletion of cubilin.

### Targeted Intracellular Delivery of a Payload Through the LRP-1 Pathway:

We synthesized HB2108, by linking AcPQLPF to Cy5 via a cathepsin-labile valine-citrulline linker. NRK cells were then treated with HB2108 and α₂M in the presence and absence of a cathepsin B inhibitor. In the absence of the inhibitor (FIG. 13A) the Cy5 staining is very diffuse and no longer colocalizes with α₂M, indicating that the chimeric molecule is cleaved thereby allowing the Cy5 dye to escape the lysosome. Upon pre-treatment with a cathepsin inhibitor (FIG. 13B), the Cy5 dye remains strongly colocalized with α₂M in the lysosome, showing that the chimeric molecule requires cleavage prior to release of the Cy5 payload into the cell.

Structure of imaging agents:

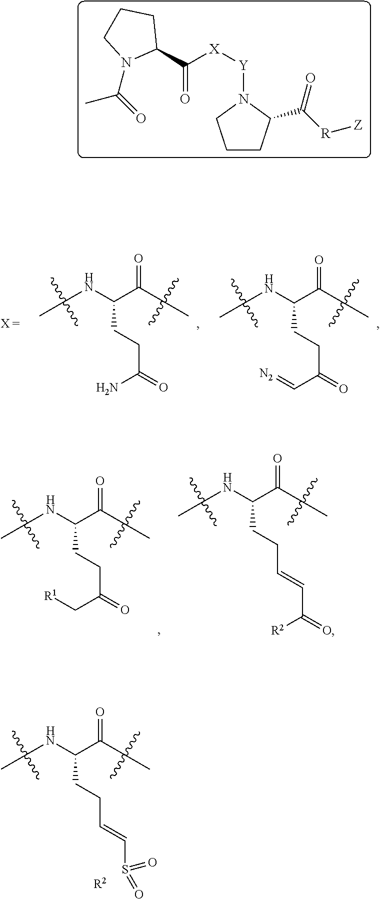

R¹═Cl, Br, I, OSO₂CF₃ etc.
R²═NMe₂, OMe, NH₂, Me, etc.
Y=Leu OR fluorophore (eg. sulfo-cyanine5, fluorescein, rhoadmine, etc.)
R=Aromatic amino acid (eg. phenylalanine, tyrosine, tryptophan, etc.)
Z═NH₂ OR fluorophore (eg. sulfo-cyanine5, fluorescein, rhoadmine, etc.)

Structure of targeting composition:

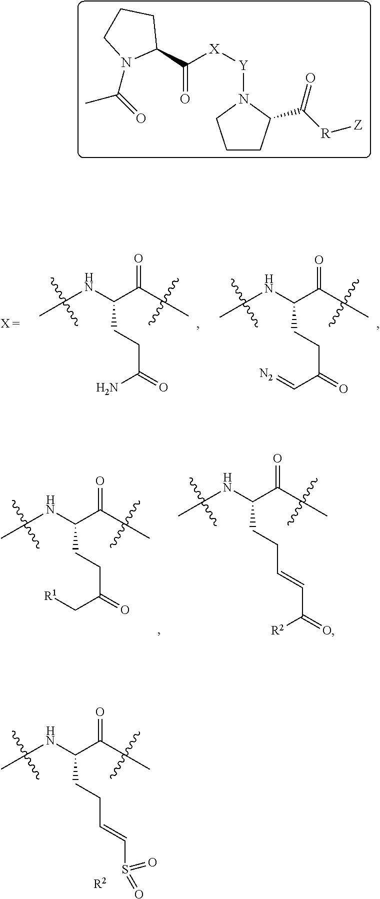

R¹=Cl, Br, I, OSO₂CF₃ etc.
R²═NMe₂, OMe, NH₂, Me, etc.
Y=Leu OR linker to small molecule ligand (eg. biotin)
R=Aromatic amino acid (eg. phenylalanine, tyrosine, tryptophan, etc.)
Z═NH₂ OR linker to small molecule ligand (eg. biotin) or linker to therapeutic protein of interest (eg. monoclonal antibody)

Structure of composition for small molecule delivery:

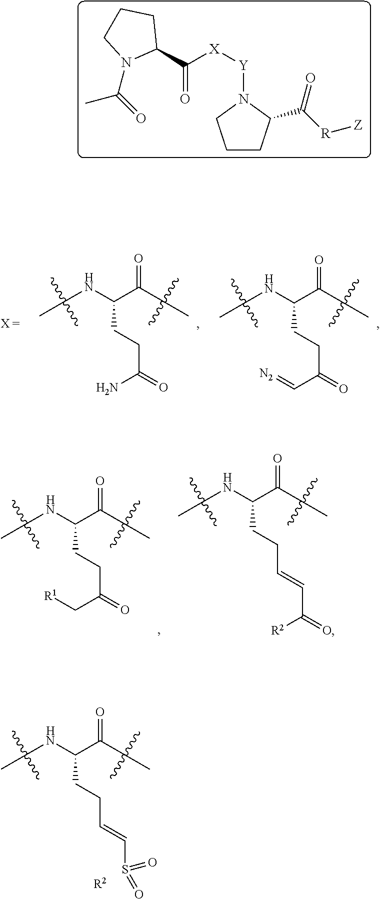

R¹=Cl, Br, I, OSO₂CF₃ etc.
R²═NMe₂, OMe, NH₂, Me, etc.
Y=Leu OR linker to small molecule payload (eg. chemotherapies)
R=Aromatic amino acid (eg. phenylalanine, tyrosine, tryptophan, etc.)
Z═NH₂ OR linker to small molecule payload (eg. chemotherapies)

### Example 2

Extensive work has intimately linked the enzymatic activity of transglutaminase 2 (TG2) to the pathogenesis of celiac disease (CeD). TG2 performs the site-selective deamidation of glutamine side chains (Gln→Glu) within wheat-derived peptides, rendering them more immunogenic in the genetic background of CeD. Additionally, recent work has shown that the amount of catalytically active TG2 in the small intestine of an individual may correlate to CeD severity. These features of TG2 have inspired the initiation of multiple drug discovery campaigns to develop novel agents to inhibit the enzyme in hopes of unlocking a novel therapeutic modality for CeD.

Herein we describe the synthesis and characterization of new small molecule probes and inhibitors of TG2 as diagnostic tools and therapeutic leads for CeD. In addition, TG2 can mediate the endolysosomal delivery of gluten antigens in the context of CeD pathogenesis. We leverage this facet of TG2 biology to bring about the delivery and/or degradation of cargos of interest to the lysosome in a TG2-dependent manner.

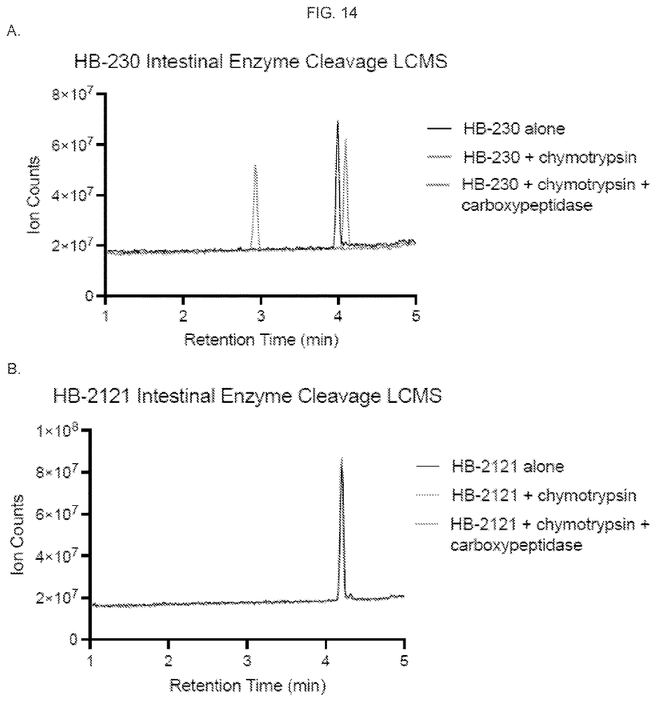

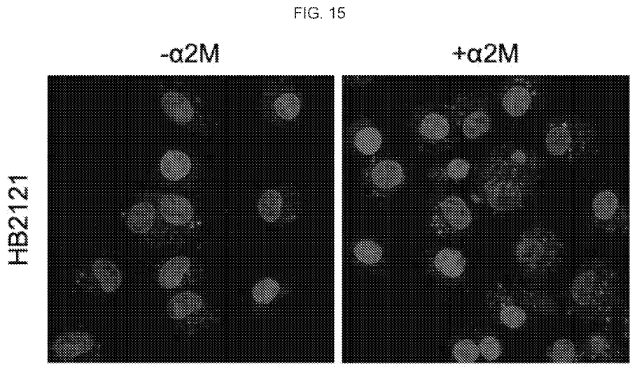

HB-2121 is analogous to the TG2 inhibitor/probe HB-230 but with one modification; the primary amide C-terminus of HB-230 was replaced with a tertiary dimethyl amide. This change greatly enhances the gastrointestinal stability of the probe. Initial studies of HB-230 revealed stability in simulated gastric and intestinal fluid as well as resistance to metabolism by the gastrointestinal enzymes pepsin, trypsin, and elastase. HB-230 suffered from enzymatic deamidation at the C-terminus (CONH₂→CO₂H) by chymotrypsin and subsequent cleavage of the C-terminal phenylalanine by carboxypeptidase. Modification of the C-terminus of HB-230 to give HB-2121 allowed complete resistance to metabolism by chymotrypsin and thus no subsequent metabolism by carboxypeptidase, shown in FIG. 14.

### Structure of HB-2121

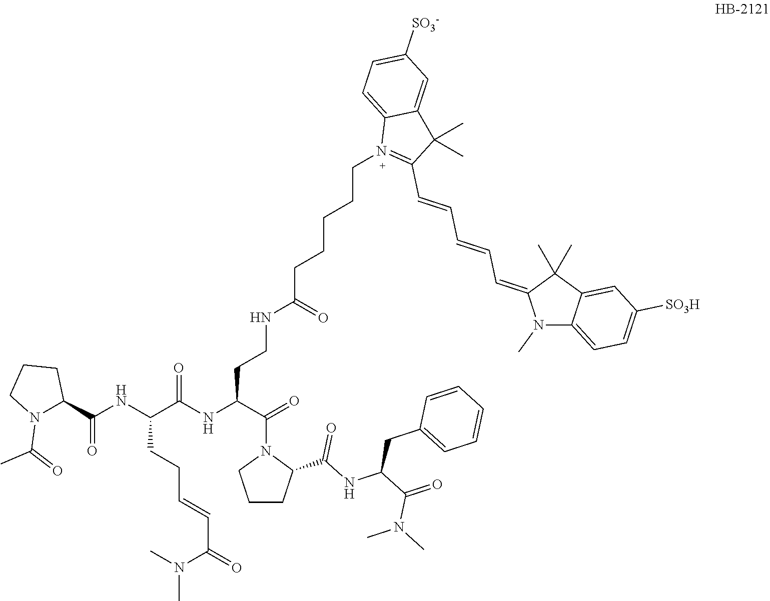

As previously described, TG2-inhibitor complexes are potently endocytosed in an α-2-macrogloblulin-dependent manner by the LRP-1 receptor. This endocytic mechanism represents a novel avenue by which cargoes of interest can be shuttled into the lysosome for delivery or degradation. Towards this end, we have designed a bifunctional small molecule (HB328) that on one end engages TG2 through covalent inhibition and on the other non-covalently binds the Fc region of IgG antibodies. The TG2 engaging half features the same pentapeptide scaffold as in HB-230. The Fc binder consists of a doubly disulfide linked 15 amino acid bicyclic peptide that has been previously characterized by Gong et al. (2016) Bioconjugate Chem. 2016, 27 (7), 1569-1573. The bifunctional molecule binds to IgG with affinity KD=3 nM.

### Structure of HB328

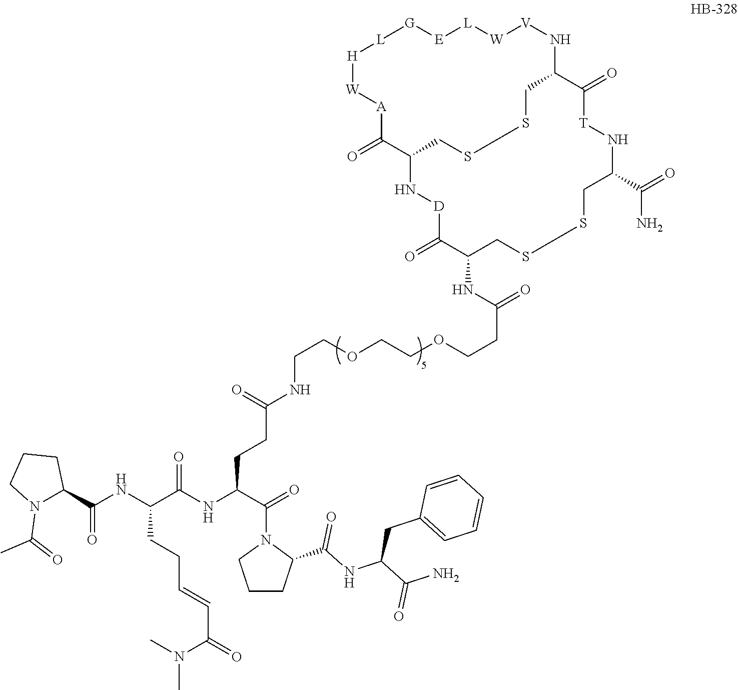

The ability of this compound to bring about endocytosis of any IgG gives it significant modularity in both a research and therapeutic context. One such therapeutic use is in the removal of maladaptive IgG that occurs in the context of rheumatologic disease (eg. rheumatoid arthritis, idiopathic thrombocytopenic purpura, etc.). In these diseases, immune complexes of IgG and antigen build up in the vessels of patients. Current treatment for the removal of such complexes relies on apheresis techniques including extracorporeal immunoadsorption where IgG is removed from a patient's blood by running the plasma over an antibody-binding column ex vivo before returning the depleted plasma back into the patient. An alternative approach involves treatment with a compound like HB-328 that allows for in vivo clearance of IgG through the TG2-LRP1 pathway described herein.

HB-328 can allow the lysosomal delivery of recombinant proteins by co-dosing HB-328 with the protein of interest, linked to an IgG Fc domain. This allows treatment of disorders of endolysosomal metabolism such as the lysosomal storage diseases.

### REFERENCES

Abadie, V., Kim, S. M., Lejeune, T., Palanski, B. A., Ernest, J. D., Tastet, O., Voisine, J., Discepolo, V., Marietta, E. v., Hawash, M. B. F., et al. (2020). IL-15, gluten and HLA-DQ8 drive tissue destruction in coeliac disease. Nature 2020 578:7796 578, 600-604. https://doi.org/10.1038/s41586-020-2003-8.
Chen, J., You, H., Li, Y., Xu, Y., He, Q., and Harris, R. C. (2018). EGF Receptor-Dependent YAP Activation Is Important for Renal Recovery from AKI. Journal of the American Society of Nephrology 29, 2372-2385. https://doi.org/10.1681/ASN.2017121272.
Falasca, L., Farrace, M. G., Rinaldi, A., Tuosto, L., Melino, G., and Piacentini, M. (2008). Transglutaminase Type II Is Involved in the Pathogenesis of Endotoxic Shock. The Journal of Immunology 180, 2616-2624. https://doi.org/10.4049/jimmunol.180.4.2616.
Ganrot, P. O., and Scherstén, B. (1967). Serum α2-macroglobulin concentration and its variation with age and sex. Clinica Chimica Acta 15, 113-120. https://doi.org/10.1016/0009-8981(67) 90333-6.
Hausch, F., Halttunen, T., Maki, M., and Khosla, C. (2003). Design, Synthesis, and Evaluation of Gluten Peptide Analogs as Selective Inhibitors of Human Tissue Transglutaminase. Chemistry & Biology 10, 225-231.
Iismaa, S. E., Mearns, B. M., Lorand, L., and Graham, R. M. (2009). Transglutaminases and Disease: Lessons From Genetically Engineered Mouse Models and Inherited Disorders. Physiological Reviews 89, 991-1023. https://doi.org/10.1152/physrev.00044.2008.
Keillor, J. W., Apperley, K. Y. P., and Akbar, A. (2015). Inhibitors of tissue transglutaminase. Trends in Pharmacological Sciences 36, 32-40. https://doi.org/10.1016/j.tips.2014.10.014. Kim, C.-Y., Quarsten, H., Bergseng, E., Khosla, C., and Sollid, L. M. (2004). Structural basis for HLA-DQ2-mediated presentation of gluten epitopes in celiac disease. Proceedings of the National Academy of Sciences 101, 4175-4179. https://doi.org/10.1073/pnas.0306885101.
Klöck, C., Herrera, Z., Albertelli, M., and Khosla, C. (2014). Discovery of Potent and Specific Dihydroisoxazole Inhibitors of Human Transglutaminase 2. Journal of Medicinal Chemistry 57, 9042-9064. https://doi.org/10.1021/jm501145a.
Lorand, L., and Graham, R. M. (2003). Transglutaminases: crosslinking enzymes with pleiotropic functions. Nature Reviews Molecular Cell Biology 4, 140-156. https://doi.org/10.1038/nrm1014. Nurminskaya, M. v., and Belkin, A. M. (2012). Cellular Functions of Tissue Transglutaminase.pp. 1-97.
Palanski, B. A., and Khosla, C. (2018). Cystamine and Disulfiram Inhibit Human Transglutaminase 2 via an Oxidative Mechanism. Biochemistry 57, 3359-3363. https://doi.org/10.1021/acs.biochem.8b00204.
Pinkas, D. M., Strop, P., Brunger, A. T., and Khosla, C. (2007). Transglutaminase 2 Undergoes a Large Conformational Change upon Activation. PLoS Biology 5, e327. https://doi.org/10.1371/journal.pbio.0050327.
Shan, L., Molberg, ø., Parrot, I., Hausch, F., Filiz, F., Gray, G. M., Sollid, L. M., and Khosla, C. (2002). Structural Basis for Gluten Intolerance in Celiac Sprue. Science (1979) 297, 2275-2279.
Matic, G.; Bosch, T.; Ramlow, W. Background and Indications for Protein A-Based Extracorporeal Immunoadsorption. Therapher Dial 2001, 5 (5), 394-403.
Balint, J. P. Immune Modulation Associated with Extracorporeal Immunoadsorption Treatments Utilizing Protein A/Silica Columns. Artificial Organs 1996, 20 (8), 906-913.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method of delivering a molecular cargo to endo-lysosomal compartments of LRP1-expressing cells, the method comprising linking the molecular cargo to a TG2 substrate or inhibitor, comprising a pentapeptide sequence Pro-X-Y-Pro-R,
where X is selected from amino acids that engage TG2 through the formation of a covalent enzyme-compound intermediate, optionally glutamine; Y is any amino acid; R is a natural or non-natural aromatic amino acid; wherein the molecular cargo is linked to the C-terminus of the pentapeptide, and/or to Y;
and contacting the LRP1-expressing cell with the TG2 substrate or inhibitor linked to the molecular cargo.

2. The method of claim 1, wherein the TG2 substrate/inhibitor comprises: 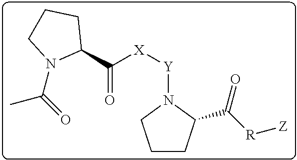
where X is selected from: where X is selected from: 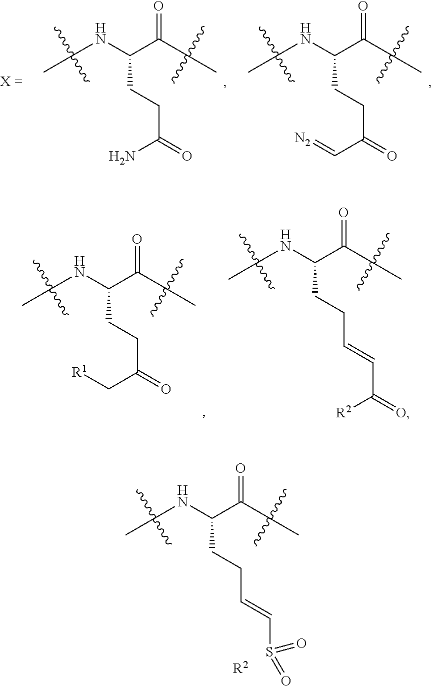
where R₁ is selected from Cl, Br, I, OSO₂CF₃; R₂ is selected from N(CH₃)₂, OCH₃, NH₂, and CH₃;
Y is any amino acid, 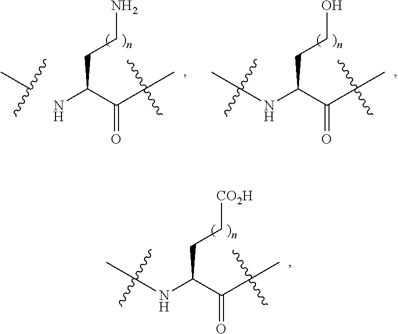
molecular cargo, or linker, optionally where Y is leu (L);
R is an aryl, heteroaryl or substituted aryl group optionally where R is Ph, CH₂Ph, CH₂PhOH, CH₂, naphthalene;
Z is OH, OR₃, NH₂, NHR₃, or Z is molecular cargo or a linker; and
R₃ is an alkyl.

3. The method of claim 1, wherein the molecular cargo comprises a bifunctional linker.

4. The method of claim 1, wherein the molecular cargo comprises a linker cleavable in the lysosome.

5. The method of claim 1, wherein the molecular cargo is an imaging moiety.

6. The method of claim 1, wherein the molecular cargo is a targeting moiety.

7. The method of claim 1, wherein the molecular cargo is a therapeutic moiety.

8. A TG2 substrate comprising or consisting of: a pentapeptide sequence Pro-X—Y-Pro-Φ,
where X is selected from amino acids that engage TG2 through the formation of a covalent enzyme-compound intermediate, optionally glutamine; Y is any amino acid; Φ is a natural or non-natural aromatic amino acid; comprising molecular cargo or a linker joined to the C-terminus of the pentapeptide, and/or to Y.

9. The TG2 substrate/inhibitor of claim 8, comprising the formula: 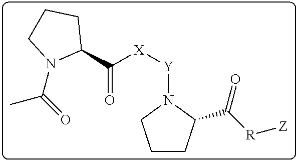
where X is selected from: where X is selected from: 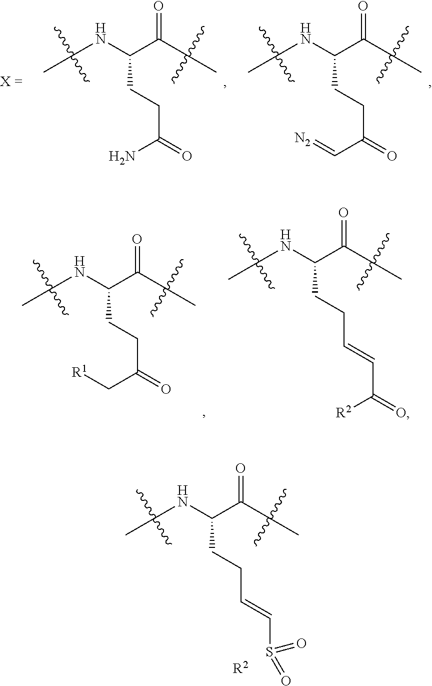
where R₁ is selected from CI, Br, I, OSO₂CF₃;
R² is selected from N(CH₃)₂, OCH₃, NH₂, CH₃;
Y is any amino acid, 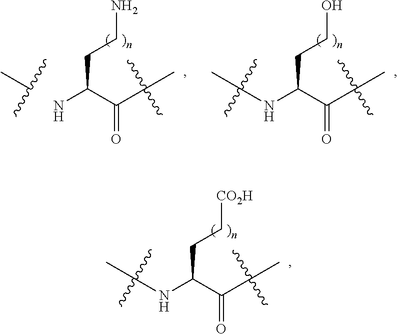
or molecular cargo, or a linker, optionally Y is leu (L);
R is an aryl, heteroaryl or substituted aryl group, optionally R is Ph, CH₂Ph, CH₂PhOH, CH₂Naphthelene and
Z is OH, OR₃, NH₂, NHR₃, or Z is molecular cargo or a linker; and R₃ is an alkyl.

10. The TG2 substrate/inhibitor of claim 7, wherein the molecular cargo comprises a bifunctional linker.

11. The TG2 substrate/inhibitor of claim 7, wherein the molecular cargo comprises a linker cleavable in the lysosome.

12. The TG2 substrate/inhibitor of claim 7, wherein the molecular cargo is an imaging moiety.

13. The TG2 substrate/inhibitor of claim 12, wherein the inhibitor is:

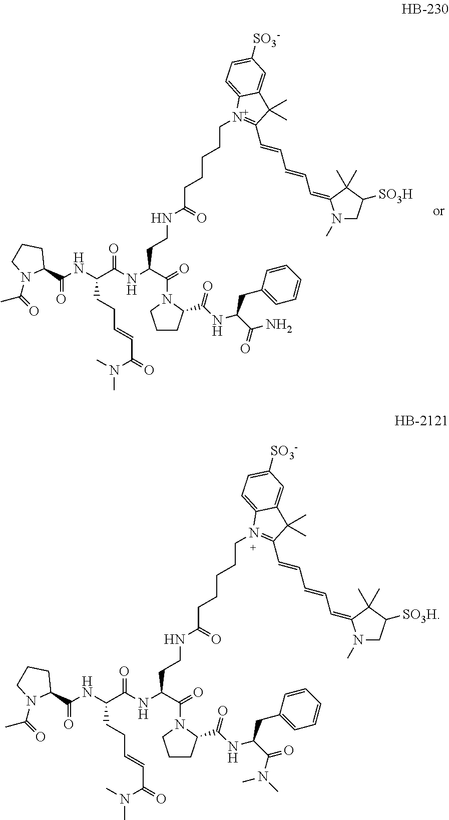

14. The TG2 substrate/inhibitor of claim 7, wherein the molecular cargo is a targeting moiety.

15. The TG2 substrate/inhibitor of claim 7, wherein the molecular cargo is a therapeutic moiety.

16. The TG2 substrate/inhibitor of claim 7, wherein the molecular cargo is an IgG binding moiety.

17. The TG2 substrate/inhibitor of claim 16, wherein the IgG-binding moiety is complexed with an IgG protein.

18. The TG2 substrate/inhibitor of claim 17, wherein the IgG protein is fused to a polypeptide of interest.

19. The TG2 substrate/inhibitor of claim 16 of the structure: [chemical structure]
